(19) United States
(12) Patent Application Publication
(10) Pub. No.: US 2005/0208047 A1
(43) Pub. Date: Sep. 22, 2005

(54) Pain signaling molecules

(76) Inventors: David J. Anderson, Altadena, CA (US); Xinzhong Dong, Pasadena, CA (US); Mark Zylka, Pasadena, CA (US); Sang-Kyou Han, Arcadia, CA (US); Melvin Simon, Pasadena, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/117,746

(22) Filed: Apr. 27, 2005

Related U.S. Application Data
(63) Continuation of application No. 10/327,387, filed on Dec. 20, 2002.

(51) Int. Cl. C12Q 1/68; C07H 21/04; A61K 39/395; C12N 15/09; C07K 14/705

(52) U.S. Cl. 424/143.1; 435/6; 435/320.1; 435/69.1; 435/325; 530/350; 536/23.5

(57) ABSTRACT

A novel G protein-coupled receptor called MrgC11 has been identified that is expressed in dorsal root ganglia and that is activated by RF amide related peptides.

### REFERENCE TO RELATED APPLICATION

The present application claims priority under 35 U.S.C. §120 as a continuation application of U.S. application Ser. No. 10/327,387, filed Dec. 20, 2002, which is incorporated by reference herein.

### BACKGROUND OF THE INVENTION

1. Field of the Invention

The invention relates generally to the field of G protein coupled receptors, and particularly to a novel G protein-coupled receptor called MrgC11 that is expressed in dorsal root ganglia and that is activated by RF amide related peptides.

2. Description of the Related Art

The treatment of acute and chronic intractable pain is a major target of drug development in the pharmaceutical industry. Pain sensation is mediated by primary sensory neurons in the dorsal root ganglia (DRG), which project peripherally to the skin and centrally to the spinal cord. These neurons express signaling molecules, such as receptors, ion channels and neuropeptides, which are involved in pain sensation. One example is the so-called Vanilloid Receptor-1 (VR-1), which is activated by capsaicin (chili pepper) as well as by heat and acid. Such pain signaling molecules may also influence pain sensation indirectly by acting as positive or negative modulators of the sensory pathway. Searching for drugs that interact with such signaling molecules, for example as receptor agonists or antagonists, is an important approach to the discovery of new therapeutics for the treatment of pain. New candidate signaling molecules expressed by pain-sensing (“nociceptive”) sensory neurons are therefore highly desirable targets for new drug screening and drug discovery efforts.

While pain is usually a natural consequence of tissue injury, as the healing process commences the pain and tenderness associated with the injury resolve. However, some individuals experience pain without an obvious injury or suffer protracted pain after an initial insult. In addition, chronic or intractable pain may occur in association with certain illnesses, such as, for example, bone degenerative diseases, terminal cancer, AIDS, and Reflex sympathetic dystrophy (RSD). Such patients may be unable to receive relief with currently-available pain-relieving (anti-nociceptive) drugs, such as opioid compounds, e.g. morphine, due to problems such as dependence and tolerance. Therefore, there is a great need for novel therapeutic agents for the treatment of pain.

A novel family of g-protein coupled receptors (GPCRs) called mrgs (mas-related genes) was recently identified in mice and humans (Dong et al. Cell 16:619-632 (2001); U.S. patent application Ser. Nos. 09/849,869 and 09/704,707). The family has been divided into three major homology groups MrgA, MrgB and MrgC and is comprised of at least 32 murine and 4 human genes (hMrgX1-hMrgX4) with intact coding sequences and additional related pseudogenes (Dong et al., supra; Simonin et al. Nat. Neurosci. 5:185-186 (2002)). Several of these receptors, including MrgA1, MrgA4 and MAS1 have been shown to be distinctively activated by RF-amide (RFa) neuopeptides, of which the prototypic member is the molluscan peptide FMRF-amide (FMRFa;).

The FMRFa-related peptides constitute a large family of neuropeptides that are widely and abundantly distributed in invertebrates, functioning as neurotransmitters and neuromodulators (Greenberg et al. Prog. Brain. Res. 92: 25-37 (1992); Li et al. Brain Res. 848:26-34 (1999)). In vertebrates only a few RFa peptides have been identified, including NPFF and NPAF (Perry et al. FEBS Lett. 409:426-430 (1997); Vilim et al. Mol. Pharmacol. 55:804-811 (1999)), the prolactin releasing peptide (Hinuma et al. Nature 393:272-276 (1998), the two RFRPs (Hinuma et al. Nat. Cell Biol. 2:703-708 (2000)), the kisspeptin (Kotani et al. J. Biol. Chem. 276:34631-34636 (2001)) and γ1-MSH. The functional significance of these peptides has been well documented (Bonini et al. J. Biol. Chem. 275:39324-39332 (2000); Ohtaki et al. Nature 411:613-617 (2001); Panula et al. Prog. Neurobiol. 48:461-487 (1996); Muir et al. J. Biol. Chem. 276:28969-28975 (2001); and Clements et al. Biochem. Biophys. Res. Commun. 284:1189-1193(2001)).

A recent study by has shown that human MrgX1 is expressed solely in dorsal root ganglia and is potently activated by the preproenkephalin products, in particular adrenal medulla peptide 22 (BAM-22P; Lembo et al. Nat. Neurosci. 5:201-209 (2002)).

### SUMMARY OF THE INVENTION

The present inventors recently carried out a screen for genes expressed in wild-type but not Ngn1^{-/-} DRG using positive selection-based differential hybridization. This screen identified both known signaling molecules involved in nociceptive neuron function, such as VR-1, and novel signaling molecules that are highly specifically expressed in nociceptive sensory neurons. In particular, the screen identified a family of G protein-coupled receptors, termed mrg for mas related genes. Subsequent experiments confirmed that mrg genes were expressed specifically in subsets of nociceptive neurons in DRG. One subfamily of Mrg's, known as MrgC, appeared to consist entirely of pseudogenes. Further experimentation has determined that one member of the MrgC family, MrgC11, is expressed and is activated by neuropeptide ligands.

In particular, the invention includes isolated nucleic acid molecules selected from the group consisting of an isolated nucleic acid molecule comprising a sequence having at least 80% sequence identity to a nucleic acid molecule that encodes the MrgC11 polypeptide with the amino acid sequence of SEQ ID NO: 2, isolated nucleic acid molecules that hybridize to the complement of a nucleic acid molecule comprising a sequence having at least 80% sequence identity to a nucleic acid molecule that encodes the MrgC11 polypeptide with the amino acid sequence of SEQ ID NO: 2, an isolated nucleic acid molecule that that hybridizes under stringent conditions to a nucleic acid molecule that encodes the MrgC1 polypeptide of SEQ ID NO:2 and an isolated nucleic acid molecule that hybridizes to the complement of a nucleic acid molecule that encodes the MrgC11 polypeptide of SEQ ID NO: 2.

The present invention also includes the nucleic acid molecules described above operably linked to one or more expression control elements, such as a promoter, as well as vectors comprising the isolated nucleic acid molecules. The invention further includes host cells transformed to contain the nucleic acid molecules of the invention and methods for producing a protein comprising the step of culturing a host cell transformed with a nucleic acid molecule of the invention under conditions in which the protein is expressed. The host cells may be prokaryotic cells, such as E. coli or eukaryotic cells, such as hamster embyonic kidney (HEK) cells or yeast cells.

The invention further provides an isolated Mrg polypeptide selected from the group consisting of isolated polypeptides encoded by the isolated nucleic acids described above and the human MrgC11 polypeptide of SEQ ID NO: 2.

The MrgC11 polypeptide may be fused to a heterologous amino acid sequence, such as an eptiope tag sequence or an immunoglobulin constant domain sequence to produce a chimeric molecule.

The invention further provides an isolated antibody that specifically binds to an MrgC11 polypeptide, including agonist and neutralizing antibodies, monoclonal and polyclonal antibodies, antibody fragments and humanized antibodies.

In another aspect, the invention provides a composition of matter comprising an MrgC11 polypeptide or an anti-MrgC11 antibody in admixture with a pharmaceutically acceptable carrier. An article of manufacture is also provided comprising the composition of matter, a container, and instructions for using the composition of matter to alter sensory perception in a mammal.

In a further aspect, the invention provides a method of identifying a compound that can be used to alter pain perception in a mammal. Test compounds are contacted with at least a portion of an MrgC11 polypeptide. The MrgC11 polypeptide or the test compound may be attached to a solid support, such as a microtiter plate. In addition, either the test compound or the MrgC11 polypeptide is preferably labeled.

Test compounds that are able to form complexes with the MrgC11 polypeptide are identified. The effects of these compounds is measured in an animal model of pain and compounds that alter pain perception in the animal model are identified as useful in altering pain perception in a mammal. The compound may enhance or decrease the perception of pain.

In one embodiment the MrgC11 polypeptide is a native MrgC11 polypeptide, preferably the MrgC11 polypeptide of SEQ ID NO: 2.

In another embodiment the MrgC11 polypeptide may be present in a cell membrane or a fraction of a cell membrane prepared from cells expressing the MrgC11 polypeptide, such as DRG cells. In a further embodiment, the MrgC11 polypeptide is present in an immunoadhesin.

The test compounds are preferably selected from the group consisting of peptides, peptide mimetics, antibodies, small organic molecules and small inorganic molecules. In a preferred embodiment the test compounds are peptides. The peptides may be anchored to a solid support by specific binding to an immobilized antibody. In addition, the test compounds may be contained in a cellular extract, particularly a cellular extract prepared from cells known to express an MrgC11 polypeptide, such as dorsal root ganglion cells.

In another aspect, the invention provides a method of identifying a compound that binds an MrgC11 polypeptide by contacting an MrgC11 polypeptide or fragment with a test compound and a ligand, such as γ2-MSH, anthoRF-amide, γ1-MSH, Dynorphin-14 or BAM22P, under conditions where binding can occur. Preferably the MrgC11 polypeptide is contacted with the peptide prior to being contacted with the test compound. The ability of the test compound to interfere with biding of the peptide to the MrgC11 polypeptide is determined.

In one embodiment the MrgC11 polypeptide is a native MrgC11 polypeptide, preferably the MrgC11 polypeptide of SEQ ID NO: 2.

The invention also provides a method of identifying an MrgC11 agonist. An MrgC11 polypeptide is expressed in a host cell capable of producing a second messenger response. In one embodiment the host cell is a eukaryotic cell, preferably a hamster embryonic kidney (HEK) cell.

The host cell is contacted with one or more test compounds and the second messenger response is measured. Compounds that increase the measured second messenger response are identified as agonists that can be used to alter sensory perception in a mammal. In one embodiment measuring the second messenger response comprises measuring a change in intercellular calcium concentration. This may be done, for example, by using a FURA-2 indicator dye. In another embodiment a second messenger response is measured by measuring the flow of current across the cell membrane.

In another aspect, the invention provides a method for identifying an MrgC11 polypeptide antagonist.

In one embodiment, an MrgC11 polypeptide, preferably the MrgC11 polypeptide of SEQ ID NO: 2, is expressed in a host cell capable of producing a second messenger response. The host cell is then contacted with a peptide ligand and one or more test compounds. The second messenger response is measured, such as by the methods described above, and compounds that alter the second messenger response to the peptide are identified as agonists.

In yet another aspect, the present invention provides a method of identifying an anti-MrgC11 agonist antibody that can be used to alter the perception of pain in a mammal. In one embodiment the method is used to identify anti-MrgC11 agonist antibodies that can be used to treat pain in a mammal.

In a preferred embodiment, candidate antibodies are prepared that specifically bind to an MrgC11 polypeptide, more preferably to the MrgC11 polypeptide of SEQ ID NO: 2. An MrgC11 polypeptide, preferably the MrgC11 polypeptide of SEQ ID NO: 2, is expressed in a host cell known to be capable of producing a second messenger response. The host cell is then contacted with a candidate antibody and the second messenger response is measured. Antibodies that increase the second messenger response are identified as agonist antibodies that can be used to treat pain in a mammal.

The invention also provides a method of treating pain in a mammal, comprising administering to the mammal an MrgC11 agonist.

### BRIEF DESCRIPTION OF THE DRAWINGS

FIG. 1A shows a sequence comparison of mouse MrgA1 (SEQ ID NO:3), MrgC11 (SEQ ID NO:2) and human MrgX1 (SEQ ID NO:4). Residues shaded in black are identical in >50% of the proteins and residues shaded in gray indicate conservative substitutions. The seven transmembrane domains (TM1-7) are over-lined. FIGS. 1B and C show in situ hybridization with cRNA riboprobes detecting mMrgC11 in newborn (FIG. 1B) and adult (FIG. 1C) DRG neurons. FIG. 1D shows double label in situ with mMrgC11 probe (red) and staining with fluorescent lectin IB4 (green) in adult mouse DRG neurons.

FIG. 2 shows calcium signaling in HEK-MrgA1 (A-C) and HEK-MrgC11 (D-F). Cells loaded with Fura-2/AM were stimulated with each agonist and fluorescence was recorded. Graphs represent an average plot of [Ca²⁺]ᵢ measurements versus time (in s) in a minimum of 8 cells from representative experiments. Individual data points represent images taken at 0.8-s intervals. FIGS. 2A and D show that U73122 (open circles), the active phospholipase C inhibitor, blocked agonis-iduced rise in [Ca²⁺]ᵢ. However, U73343 (closed circles), the inactive analog, did not affect FLRFa or γ2-MSH-induced Ca²⁺ mobilization. After a 10 minute pretreatment with U73122 and U73343, each agonist was added. FIGS. 2B and E show the extracellular [Ca²⁺] dependency of Ca²⁺ mobilization. Cells were preincubated for 2 minutes with 2 mM EGTA (open circles) or normal medium containing 1.2 mM calcium (closed circles) and then 3 μM FLRFa or 1 μM γ2-MSH was added. FIGS. 2C and F show that TG prevents the agonist-evoked increase of [Ca²⁺]ᵢ in HEK-MrgA1 (FIG. 2C) and HEK-MrgC11 (FIG. 2F). In the presence of 2 mM EGRA, TG (1 μM final concentration) was added to deplete internal Ca²⁺ stores.

FIGS. 3A-D show that internalization of MrgA1-GFP (A and B) and MrgC11-GFP (C and D) was induced by 3 μM FLRFa and 1 μM γ2-MSH, respectively. FIGS. 3A and C show serum starved (>4 hr) HEK-MrgA1 and HEK-MrgC11 cells. FIGS. 3B and D show HEK-MrgA1 or HEK-MrgC11 treated with the indicated agonists for 30 minutes at 37° C. Results are representative of three independent experiments, and the arrow indicates the internalization process.

FIGS. 4A-F show the heterotrimeric G protein coupling of MrgA1 and MrgC11. FIGS. 4A and D show that FLRFa or γ2-MSH dose-dependently stimulate intracellular calcium mobilization in HEK-MrgA1 or HEK-MrgC11 in the absence (closed circles) or presence (closed squares) of PTX (16 h, 100 ng/ml). All results shown are the mean of triplicate determination±SEM. FIGS. 4B and E show the effect of Ga subunit KO on [Ca²⁺]ᵢ mobilizatoin. KO MEFs were derived from KO mice at embryonic 8.5 and 9.5 days. Gα_{12/13} KO MEF (closed triangle) and Gα_{q/11} KO MEF (open circles) were transfected with the cDNAs encoding the MrgA1-GFP (FIG. 4B) or MrgC11-GFP (FIG. 4E). FLRFa or γ2-MSH evoked [Ca²⁺]ᵢ responses were completely abrogated in Gα_{q/11}, double KO MEF expressing MrgA1-GFP (FIG. 4B) or MrgC11-GFP (FIG. 4E). However, cotransfection (open triangles) of wild-type Gα_{q} plus MrgA1-GFP or MrgC11-GFP in Gα_{q/11} double KO MEF restored responsiveness to FLRFa or γ2-MSH, respectively. Positively transfected cells were selected by their green fluorescence excited at 480 nm (GFP-positive cells). On the same field, cells that did not express GFP (GFP-negative cells) were selected as internal control. FIGS. 4C and F show cAMP production in HEK-MrgA1 (FIG. 4C) or HEK-MrgC11 (FIG. 4F). Cells were stimulated with various concentrations fo FLRFa or γ2-MSH in the presence or absence of 10 μM forskolin. Each value represents the mean±SEM for three independent experiments.

FIG. 5 provides a nucleotide sequence (SEQ ID NO:1) encoding a native sequence murine MrgC11 (SEQ ID NO: 2)

### DETAILED DESCRIPTION OF THE PREFERRED EMBODIMENT

### I. General Description

The present invention is based in part on the discovery that MrgC11, which was initially identified as a member of a subfamily of Mrg pseudogenes, has an intact coding sequence, is expressed in a specific subpopulation of nociceptor neurons in the dorsal root ganglia (DRG), and is activated by a number of specific neuropeptides (Han et al., Proc. Natl. Acad. Sci. USA 99(23):14740-14745 (2002), incorporated herein by reference).

The Mrg family of GPCRs contains three major subfamilies (MrgA, B and C), each consisting of more than 10 highly duplicated genes, as well as several single-copy genes such as Mas1, Rta, MrgD and MrgE. Four human genes that are most closely related to the MrgA subfamily have also been identified: MrgX1; MrgX2; MrgX3; and MrgX4 (Dong et al., supra; Lembo et al., supra).

Ten members of the MrgC subfamily were initially identified in mice. However, it was believed that all members of this subfamily were pseudogenes (Dong et al., supra).

The existence of a G protein-coupled receptor specifically expressed in nociceptive sensory neurons indicates that this molecule is a primary mediator or modulator of pain sensation. It is therefore of great interest to identify ligands, both endogenous and synthetic, that modulate the activity of these receptors, for the management of pain. Indeed, ligand screens in heterologous cell expression systems indicate that MrgC11 interacts with RF-amide neuropeptides of which the prototypic member is the molluscan cardioexcitatory peptide FMRF-amide (Price and Greenberg Science 197: 670-671 (1977)). Mammalian RF-amide peptides include NPFF and NPAF, which are derived from a common pro-peptide precursor expressed in neurons of laminae I and II of the dorsal spinal cord (Vilim et al. Mol Pharmacol 55: 804-11 (1999)). The expression of this neuropeptide FF precursor in the synaptic termination zone of neurons expressing MrgC11, the ability of NPAF and NPFF to activate this receptor in functional assays, and the presence of binding sites for such peptides on primary sensory afferents in the dorsal horn (Gouarderes et al. Synapse 35: 45-52 (2000)), together indicate that these neuropeptides are ligands for MrgC11 in vivo. Intrathecal injection of NPFF/NPAF peptides produces long-lasting antinociceptive effects in several chronic pain models (reviewed in Panula et al. Brain Res 848: 191-6 (1999)), including neuropathic pain (Xu et al. Peptides 20: 1071-7 (1999)), data further indicating that MrgC is directly involved in the modulation of pain.

MrgC11 and related polypeptides described herein can serve as therapeutics and as a target for agents that modulate their expression or activity, such as for use in the treatment of chronic intractable pain and neuropathic pain. Agents may be identified which modulate biological processes associated with nociception such as the reception, transduction and transmission of pain signals.

### II. Specific Embodiments

A. Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below.

As used herein, the term “protein” or “polypeptide” refers, in part, to a protein that has the amino acid sequence depicted in SEQ ID NO: 2. The terms also refer to naturally occurring allelic variants and proteins that have a slightly different amino acid sequence than that specifically recited above. Allelic variants, though possessing a slightly different amino acid sequence than that recited above, will still have the same or similar biological functions associated with the protein.

Identity or homology with respect to amino acid sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity (see section B for the relevant parameters). Fusion proteins, or N-terminal, C-terminal or internal extensions, deletions, or insertions into the peptide sequence shall not be construed as affecting homology.

Proteins can be aligned using CLUSTALW (Thompson et al. Nucleic Acids Res 22:4673-80 (1994)) and homology or identity at the nucleotide or amino acid sequence level may be determined by BLAST (Basic Local Alignment Search Tool) analysis using the algorithm employed by the programs blastp, blastn, blastx, tblastn and tblastx (Karlin, et al. Proc. Natl. Acad. Sci. USA 87: 2264-2268 (1990) and Altschul, S. F. J. Mol. Evol. 36: 290-300 (1993), fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff, et al. Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters were adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

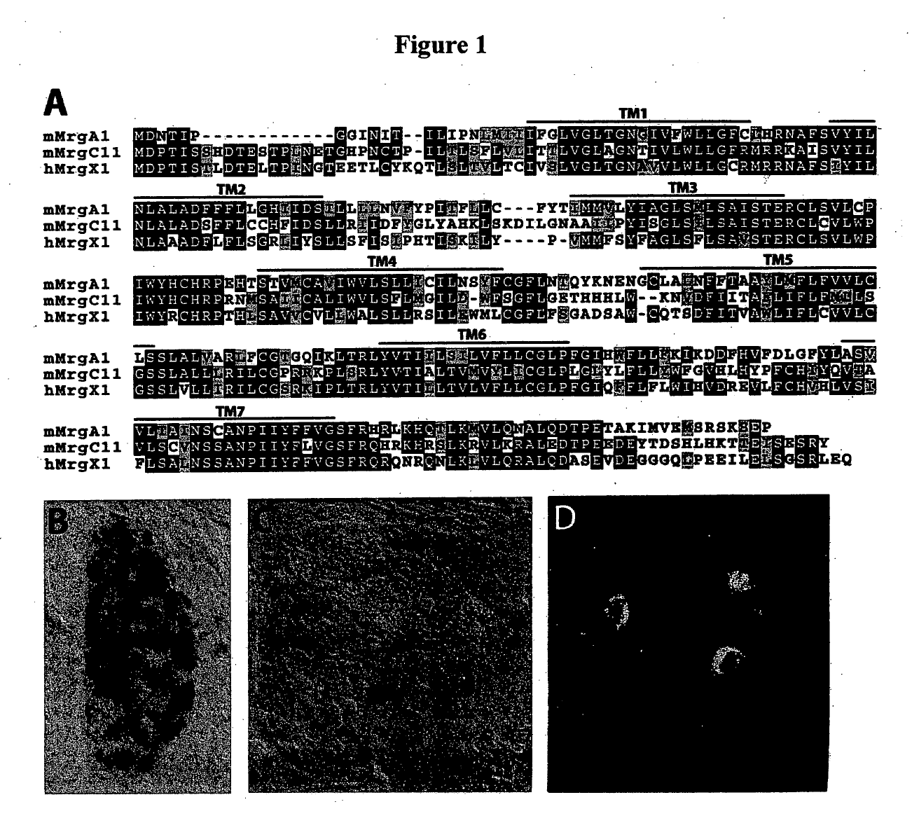

“Variants” are biologically active polypeptides having an amino acid sequence which differs from the sequence of a native sequence MrgC11 polypeptide of the present invention, such as that shown in FIG. 1 (SEQ ID NO: 2), by virtue of an insertion, deletion, modification and/or substitution of one or more amino acid residues within the native sequence. Variants include peptide fragments of at least 5 amino acids, preferably at least 10 amino acids, more preferably at least 15 amino acids, even more preferably at least 20 amino acids that retain a biological activity of the corresponding native sequence polypeptide, such as the ability to bind particular neuropeptide. Variants also include polypeptides wherein one or more amino acid residues are added at the N- or C-terminus of, or within, a native sequence. Further, variants also include polypeptides where a number of amino acid residues are deleted and optionally substituted by one or more different amino acid residues.

As used herein, a “conservative variant” refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein.

As used herein, the “family of proteins” related to MrgC11 includes proteins that have been isolated from the dorsal root ganglia of organisms in addition to mice. The methods used to identify and isolate other members of the family of proteins, such as the disclosed mouse protein, are described below.

Unless indicated otherwise, the term “MrgC11” when used herein includes native sequence mammalian, such as murine or human, MrgC11, MrgC11 variants; MrgC11 receptor extracellular domain; and chimeric MrgC11 receptors (each of which is defined herein). The term specifically includes native sequence murine MrgC11 receptors, such as SEQ ID NO: 2 and their human homologues.

The terms “mas-related gene”, “mrg” and “Mrg” are used interchangeably herein.

A “native” or “native sequence” MrgC11 receptor has the amino acid sequence of a naturally occurring MrgC11 receptor in any mammalian species (including humans), irrespective of its mode of preparation. Accordingly, a native or native sequence MrgC11 receptor may be isolated from nature, produced by techniques of recombinant DNA technology, chemically synthesized, or produced by any combinations of these or similar methods. Native MrgC11 receptors specifically include polypeptides having the amino acid sequence of naturally occurring allelic variants, isoforms or spliced variants of these receptors, known in the art or hereinafter discovered.

The “extracellular domain” (ECD) is a form of the MrgC11 receptor which is essentially free of the transmembrane and cytoplasmic domains, i.e., has less than 1% of such domains, preferably 0.5 to 0% of such domains, and more preferably 0.1 to 0% of such domains. Ordinarily, the ECD will have an amino acid sequence having at least about 60% amino acid sequence identity with the amino acid sequence of one or more of the ECDs of a native MrgC11 protein, preferably at least about 65%, more preferably at least about 75%, even more preferably at least about 80%, even more preferably at least about 90%, with increasing preference of 95%, to at least 99% amino acid sequence identity, and finally to 100% identity, and thus includes polypeptide variants as defined below.

The first predicted extracellular domain (ECD1) of MrgC11 (SEQ ID NO: 2) comprises approximately amino acids 83-104, the second predicted extracellular domain (ECD2) comprises approximately amino acids 164-175, and the third predicted ECD comprises approximately amino acids 234-257. Cytoplasmic domains are located at approximately amino acids 55-61, 124-142, 197-216 and 279 through the C terminus. Transmembrane domains are located at approximately amino acids 35-54 (TM1), 62-82 (TM2), 105-123 (TM3), 143-163 (TM4), 176-196 (TM5), 217-233 (TM6) and 258-278 (TM7). The N-terminus is predicted to be extracellular and to comprise approximately amino acids 1 through 34.

As used herein, “nucleic acid” is defined as RNA or DNA that encodes a protein or peptide as defined above, is complementary to a nucleic acid sequence encoding such peptides, hybridizes to such a nucleic acid and remains stably bound to it under appropriate stringency conditions, exhibits at least about 50%, 60%, 70%, 75%, 85%, 90% or 95% nucleotide sequence identity across the open reading frame, or encodes a polypeptide sharing at least about 50%, 60%, 70% or 75% sequence identity, preferably at least about 80%, and more preferably at least about 85%, and even more preferably at least about 90 or 95% or more identity with the peptide sequences. Specifically contemplated are genomic DNA, cDNA, mRNA and antisense molecules, as well as nucleic acids based on alternative backbones or including alternative bases whether derived from natural sources or synthesized. Such hybridizing or complementary nucleic acids, however, are defined further as being novel and unobvious over any prior art nucleic acid including that which encodes, hybridizes under appropriate stringency conditions, or is complementary to nucleic acid encoding a protein according to the present invention.

As used herein, the terms nucleic acid, polynucleotide and nucleotide are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

The terms nucleic acid, polynucleotide and nucleotide also specifically include nucleic acids composed of bases other than the five biologically occurring bases (adenine, guanine, thymine, cytosine and uracil). For example, a polynucleotide of the invention might contain at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyl-uracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Furthermore, a polynucleotide used in the invention may comprise at least one modified sugar moiety selected from the group including but not limited to arabinose, 2-fluoroarabinose, xylulose, and hexose.

“Stringent conditions” are those that (1) employ low ionic strength and high temperature for washing, for example, about 0.015 M NaCl/0.0015 M sodium citrate/0.1% SDS at about 50° C., or (2) employ during hybridization a denaturing agent such as formamide, for example, about 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at about 42° C. Another example is use of 50% formamide, 5×SSC (0.75M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at about 42° C., with washes at about 42° C. in 0.2×SSC and 0.1% SDS. A skilled artisan can readily determine and vary the stringency conditions appropriately to obtain a clear and detectable hybridization signal.

As used herein, a nucleic acid molecule is said to be “isolated” when the nucleic acid molecule is substantially separated from contaminant nucleic acid molecules encoding other polypeptides.

As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional region(s) of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming (see the discussion in Section H).

Highly related gene homologs are polynucleotides encoding proteins that have at least about 60% amino acid sequence identity with the amino acid sequence of a naturally occurring native sequence MrgC11, such as SEQ ID NO: 2, preferably at least about 65%, 70%, 75%, 80%, with increasing preference of at least about 85% to at least about 99% amino acid sequence identity, in 1% increments.

The term “mammal” is defined as an individual belonging to the class Mammalia and includes, without limitation, humans, domestic and farm animals, and zoo, sports, or pet animals, such as sheep, dogs, horses, cats or cows. Preferably, the mammal herein is human.

“Functional derivatives” include amino acid sequence variants, and covalent derivatives of the native polypeptides as long as they retain a qualitative biological activity of the corresponding native polypeptide.

By “MrgC11 ligand” is meant a molecule which specifically binds to and preferably activates an MrgC11 receptor. Examples of MrgC11 ligands include, but are not limited to γ2-MSH, γ1-MSH, BAM-22P, Dynorphin14, BAM-15, NPFF, Kiss, other peptides indicated in Table 1 below, and other neuropeptides terminating with RF(Y)G or RF(Y)a. The ability of a molecule to bind to MrgC11 can be determined, for example, by the ability of the putative ligand to bind to membrane fractions prepared from cells expressing MrgC11.

A “chimeric” molecule is a polypeptide comprising a full-length polypeptide of the present invention, a variant, or one or more domains of a polypeptide of the present invention fused or bonded to a heterologous polypeptide. The chimeric molecule will generally share at least one biological property in common with a naturally occurring native sequence polypeptide. An example of a chimeric molecule is one that is epitope tagged for purification purposes. Another chimeric molecule is an immunoadhesin.

The term “epitope-tagged” when used herein refers to a chimeric polypeptide comprising MrgC11 fused to a “tag polypeptide”. The tag polypeptide has enough residues to provide an epitope against which an antibody can be made, yet is short enough such that it does not interfere with the biological activity of MrgC11. The tag polypeptide preferably is fairly unique so that the antibody against it does not substantially cross-react with other epitopes. Suitable tag polypeptides generally have at least six amino acid residues and usually between about 8 and about 50 amino acid residues (preferably between about 9 and about 30 residues). Preferred are poly-histidine sequences, which bind nickel, allowing isolation of the tagged protein by Ni-NTA chromatography as described (See, e.g., Lindsay et al. Neuron 17:571-574 (1996)).

“Agonists” are molecules or compounds that stimulate one or more of the biological properties of a polypeptide of the present invention. These may include, but are not limited to, small organic and inorganic molecules, peptides, peptide mimetics and agonist antibodies.

The term “antagonist” is used in the broadest sense and refers to any molecule or compound that blocks, inhibits or neutralizes, either partially or fully, a biological activity mediated by a receptor of the present invention by preventing the binding of an agonist. Antagonists may include, but are not limited to, small organic and inorganic molecules, peptides, peptide mimetics and neutralizing antibodies.

The polypeptides of the present invention are preferably in isolated form. As used herein, a polypeptide is said to be isolated when physical, mechanical or chemical methods are employed to remove the polypeptide from cellular constituents with which it is normally associated. A skilled artisan can readily employ standard purification methods to obtain an isolated polypeptide. In some instances, isolated polypeptides will have been separated or purified from many cellular constituents, but will still be associated with other cellular constituents, such as cellular membrane fragments.

Thus, “isolated MrC11” means MrgC11 polypeptide that has been purified from a protein source or has been prepared by recombinant or synthetic methods and purified. Purified MrgC11 is substantially free of other polypeptides or peptides. “Substantially free” here means less than about 5%, preferably less than about 2%, more preferably less than about 1%, even more preferably less than about 0.5%, most preferably less than about 0.1% contamination with other polypeptides.

“Essentially pure” protein means a composition comprising at least about 90% by weight of the protein, based on total weight of the composition, preferably at least about 95% by weight, more preferably at least about 90% by weight, even more preferably at least about 95% by weight. “Essentially homogeneous” protein means a composition comprising at least about 99% by weight of protein, based on total weight of the composition.

“Biological property” is a biological or immunological activity, where biological activity refer to a biological function (either inhibitory or stimulatory) caused by a native sequence or variant polypeptide, other than the ability to induce the production of an antibody against an epitope within such polypeptide, where the latter property is referred to as immunological activity. Biological properties specifically include the ability to bind a naturally occurring ligand of the receptor molecules herein, preferably specific binding, and even more preferably specific binding with high affinity. For example, a biological activity of MrgC11 is the ability to bind and/or be activated by neuropeptides as described in the Examples below. A particular biological activity is release of intracellular free calcium within a cell upon activation of MrgC11 by γ2-MSH.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. Antibodies to MrgC11 preferably recognize an epitope that is unique to MrgC11.

“Native antibodies” and “native immunoglobulins” are usually heterotetrameric glycoproteins, composed of two identical light (L) chains and two identical heavy (H) chains. Each light chain is linked to a heavy chain by one covalent disulfide bond while The number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “antibody” herein is used in the broadest sense and specifically covers human, non-human (e.g. murine) and humanized monoclonal antibodies (including full length monoclonal antibodies), polyclonal antibodies, multi-specific antibodies (e.g., bispecific antibodies), and antibody fragments so long as they exhibit the desired biological activity.

“Antibody fragments” comprise a portion of a full-length antibody, generally the antigen binding or variable domain thereof. Examples of antibody fragments include Fab, Fab′, F(ab′)₂, and Fv fragments; diabodies; linear antibodies; single-chain antibody molecules; and multi-specific antibodies formed from antibody fragments.

The term “monoclonal antibody” as used herein refers to an antibody obtained from a population of antibodies wherein the individual antibodies comprising the population are identical except for possible naturally occurring mutations that may be present in minor amounts. Monoclonal antibodies are highly specific and are directed against a single antigenic site. In addition, monoclonal antibodies may be made by any method known in the art. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass. Fragments of chimeric antibodies are also included provided they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are antibodies that contain minimal sequence derived from non-human immunoglobulin. Humanized antibodies are generally human immunoglobulins in which hypervariable region residues are replaced by hypervariable region residues from a non-human species such as mouse, rat, rabbit or non-human primate having the desired specificity, affinity, and capacity. Framework region (FR) residues of the human immunoglobulin may be replaced by corresponding non-human residues. In addition, humanized antibodies may comprise residues that are not found in either the recipient antibody or in the donor antibody. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

The term “epitope” is used to refer to binding sites for (monoclonal or polyclonal) antibodies on protein antigens.

By “agonist antibody” is meant an antibody which is a ligand for MrgC11 and thus is able to activate and/or stimulate one or more of the effector functions and/or biological acitivities of native sequence MrgC11.

By “neutralizing antibody” is meant an antibody molecule as herein defined which is able to block or significantly reduce an effector function and/or biological acitivity of a polypeptide of the invention. For example, a neutralizing antibody may inhibit or reduce MrgC11 activation by a known ligand.

The term “MrgC11 immunoadhesin” refers to a chimeric molecule that comprises at least a portion of an MrgC11 molecule (native or variant) and an immunoglobulin sequence. The immunoglobulin sequence preferably, but not necessarily, is an immunoglobulin constant domain. Immunoadhesins can possess many of the properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. If the two arms of the immunoadhesin structure have different specificities, the immunoadhesin is called a “bispecific immunoadhesin” by analogy to bispecific antibodies.

As used herein, “treatment” is a clinical intervention made in response to a disease, disorder or physiological condition manifested by a patient. The aim of treatment includes the alleviation or prevention of symptoms, slowing or stopping the progression or worsening of a disease, disorder, or condition and the remission of the disease, disorder or condition. “Treatment” refers to both therapeutic treatment and prophylactic or preventative measures. Those in need of treatment include those already affected by a disease or disorder or undesired physiological condition as well as those in which the disease or disorder or undesired physiological condition is to be prevented. Specifically, treatment may alleviate pain, including pain resulting from an existing condition or disorder, or to prevent pain in situations where pain is likely to be experienced.

In the methods of the present invention, the term “control” and grammatical variants thereof, are used to refer to the prevention, partial or complete inhibition, reduction, delay or slowing down of an unwanted event, such as the presence or onset of pain.

The term “effective amount” refers to an amount sufficient to effect beneficial or desirable clinical results. An effective amount of an agonist or antagonist is an amount that is effective to treat a disease, disorder or unwanted physiological condition.

“Pain” is a sensory experience perceived by nerve tissue distinct from sensations of touch, pressure, heat and cold. The range of pain sensations, as well as the variation of perception of pain by individuals, renders a precise definition of pain impossible. In the context of the present invention, “pain” is used in the broadest possible sense and includes nociceptive pain, such as pain related to tissue damage and inflammation, pain related to noxious stimuli, acute pain, chronic pain, and neuropathic pain.

“Acute pain” is often short-lived and typically has a specific cause. Acute pain can occur, for example, during soft tissue injury and with infection and inflammation. It can be modulated and removed by treating its cause and through combined strategies, for example using analgesics to treat the pain and antibiotics to treat an infection.

“Chronic pain” is distinctly different from and more complex than acute pain. Chronic pain has no time limit, often has no apparent cause and may serve no apparent biological purpose. Chronic pain can trigger multiple psychological problems that confound both patient and health care provider, leading to feelings of helplessness and hopelessness. The most common types of chronic pain include low-back pain, headache, recurrent facial pain, pain associated with cancer and arthritis pain.

Pain is termed “neuropathic” when it is taken to be representative of neurologic dysfunction. “Neuropathic pain” typically has a complex and variable etiology. It may be characterized by hyperalgesia (lowered pain threshold and enhanced pain perception) and by allodynia (pain from innocuous mechanical or thermal stimuli). Neuropathic pain is usually chronic and tends not to respond to the same drugs as “normal pain” (nociceptive pain). Therefore, its treatment is often much more difficult than the treatment of nociceptive pain.

Neuropathic pain may develop whenever nerves are damaged, for example by trauma, by disease such as diabetes, herpes zoster, or late-stage cancer, or by chemical injury (e.g., as an untoward consequence of therapeutic agents including the false-nucleotide anti-HIV drugs). It may also develop after amputation (including mastectomy). Examples of neuropathic pain include monoradiculopathies, trigeminal neuralgia, postherpetic neuralgia, complex regional pain syndromes and the various peripheral neuropathies. This is in contrast with “normal pain” or “nociceptive pain,” which includes normal post-operative pain, pain associated with trauma, and chronic pain of arthritis.

“Peripheral neuropathy” is a neurodegenerative disorder that affects the peripheral nerves, most often manifested as one or a combination of motor, sensory, sensorimotor, or autonomic dysfunction. Peripheral neuropathies may, for example, be characterized by the degeneration of peripheral sensory neurons, which may result from a disease or disorder such as diabetes (diabetic neuropathy), alcoholism and acquired immunodeficiency syndrome (AIDS), from therapy such as cytostatic drug therapy in cancer, or from genetic predisposition. Genetically acquired peripheral neuropathies include, for example, Krabbe's disease, Metachromatic leukodystrophy, and Charcot-Marie-Tooth (CMT) Disease. Peripheral neuropathies are often accompanied by pain.

“Pharmaceutically acceptable” carriers, excipients, or stabilizers are ones which are nontoxic to the cell or mammal being exposed thereto at the dosages and concentrations employed. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™.

“Peptide mimetics” are molecules which serve as substitutes for peptides in interactions with the receptors of the present invention (Morgan et al., Ann. Reports Med. Chem. 24:243-252 (1989)). Peptide mimetics, as used herein, include synthetic structures that retain the structural and functional features of a peptide. Peptide mimetics may or may not contain amino acids and/or peptide bonds. The term, “peptide mimetics” also includes peptoids and oligopeptoids, which are peptides or oligomers of N-substituted amino acids (Simon et al., Proc. Natl. Acad. Sci. USA 89:9367-9371 (1972)). Further included as peptide mimetics are peptide libraries, which are collections of peptides designed to be of a given amino acid length and representing all conceivable sequences of amino acids corresponding thereto.

A. Proteins Expressed in Primary Sensory Neurons of Dorsal Root Ganglia

In one aspect the present invention provides isolated MrgC11 proteins, allelic variants thereof, and proteins comprising conservative amino acid substitutions. A polypeptide sequence of murine MrgC11 is provided in SEQ ID NO: 2.

The proteins of the present invention further include insertion, deletion or conservative amino acid substitution variants of the sequence set forth in SEQ ID NO: 2.

Ordinarily, the variants, allelic variants, the conservative substitution variants, and the members of the protein family, including corresponding homologues in other species, will have an amino acid sequence having at least about 50%, or about 60% to 75% amino acid sequence identity with the sequence set forth in SEQ ID NO: 2, more preferably at least about 80%, even more preferably at least about 90%, and most preferably at least about 95% sequence identity with said sequences.

The proteins of the present invention include molecules having the amino acid sequence disclosed in SEQ ID NO: 2, fragments thereof having a consecutive sequence of at least about 3, 4, 5, 6, 10, 15, 20, 25, 30, 35 or more amino acid residues of the protein, amino acid sequence variants wherein one or more amino acid residues has been inserted N- or C-terminal to, or within, the disclosed coding sequence, and amino acid sequence variants of the disclosed sequence, or their fragments as defined above, that have been substituted by another residue. Such fragments, also referred to as peptides or polypeptides, may contain antigenic regions, functional regions of the protein identified as regions of the amino acid sequence which correspond to known protein domains, as well as regions of pronounced hydrophilicity. The regions are all easily identifiable by using commonly available protein sequence analysis software such as MACVECTOR™ (Oxford Molecular).

Contemplated variants further include those containing predetermined mutations by, e.g., homologous recombination, site-directed or PCR mutagenesis, and the corresponding proteins of other animal species, including but not limited to rabbit, rat, porcine, bovine, ovine, equine, human and non-human primate species, and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

Protein domains such as a ligand binding domain, an extracellular domain, a transmembrane domain (e.g. comprising seven membrane spanning segments and cytosolic loops or two membrane spanning domains and cytosolic loops), the transmembrane domain and a cytoplasmic domain and an active site may all be found in the proteins or polypeptides of the invention. Such domains are useful for making chimeric proteins and for in vitro assays of the invention.

Variations in native sequence proteins of the present invention or in various domains identified therein, can be made, for example, using any techniques known in the art. Variation can be achieved, for example, by substitution of at least one amino acid with any other amino acid in one or more of the domains of the protein. A change in the amino acid sequence of a protein of the invention as compared with a native sequence protein may be produced by a substitution, deletion or insertion of one or more codons encoding the protein. A comparison of the sequence of the MrgC11 polypeptide to be changed with that of homologous known protein molecules may provide guidance as to which amino acid residues may be inserted, substituted or deleted without affecting a desired biological activity. In particular, it may be beneficial to minimize the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Polypeptide fragments are also provided and are useful in the methods of the present invention. Such fragments may be truncated at the N-terminus or C-terminus, or may lack internal residues, for example, when compared with a full-length native protein. Certain fragments lack amino acid residues that are not essential for a desired biological activity of the MrgC11 polypeptide.

MrgC11 fragments may be prepared by any of a number of conventional techniques. Desired peptide fragments may be chemically synthesized or generated by enzymatic digestion, such as by treating the protein with an enzyme known to cleave proteins at sites defined by particular amino acid residues. Alternatively, the DNA encoding the protein may be digested with suitable restriction enzymes and the desired fragment isolated. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, MrgC11 polypeptide fragments share at least one biological and/or immunological activity with a native MrgC11 polypeptide.

In making amino acid sequence variants that retain the required biological properties of the corresponding native sequences, the hydropathic index of amino acids may be considered. For example, it is known that certain amino acids may be substituted for other amino acids having a similar hydropathic index or score without significant change in biological activity. Thus, isoleucine, which has a hydropathic index of +4.5, can generally be substituted for valine (+4.2) or leucine (+3.8), without significant impact on the biological activity of the polypeptide in which the substitution is made. Similarly, usually lysine (−3.9) can be substituted for arginine (−4.5), without the expectation of any significant change in the biological properties of the underlying polypeptide. Other considerations for choosing amino acid substitutions include the similarity of the side-chain substituents, for example, size, electrophilic character, charge in various amino acids. In general, alanine, glycine and serine; arginine and lysine; glutamate and aspartate; serine and threonine; and valine, leucine and isoleucine are interchangeable, without the expectation of any significant change in biological properties. Such substitutions are generally referred to as conservative amino acid substitutions, and are the preferred type of substitutions within the polypeptides of the present invention.

Non-conservative substitutions will entail exchanging a member of one class of amino acids for another class. Such substituted residues also may be introduced into the conservative substitution sites or, more preferably, into the remaining (non-conserved) sites.

The variations can be made using methods known in the art such as site-directed mutagenesis, alanine scanning mutagenesis, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on cloned DNA to produce the MrgC11 variant DNA.

Scanning amino acid analysis can be employed to identify one or more amino acids that can be replaced without a significant impact on biological activity. Among the preferred scanning amino acids are relatively small, neutral amino acids. Such amino acids include alanine, glycine, serine, and cysteine. Alanine is preferred because, in addition to being the most common amino acid, it eliminates the side-chain beyond the beta-carbon and is therefore less likely to alter the main-chain conformation of the variant (Cunningham and Wells, Science, 244: 1081-1085 (1989)). Further, alanine is frequently found in both buried and exposed positions (Creighton, The Proteins, (W.H. Freeman & Co., N.Y.); Chothia, J. Mol. Biol., 150:1 (1976)). If alanine substitution does not yield adequate amounts of variation, an isoteric amino acid can be used.

As described below, members of the family of proteins can be used: 1) to identify agents which modulate at least one activity of the protein; 2) to identify binding partners for the protein, 3) as an antigen to raise polyclonal or monoclonal antibodies, 4) as a therapeutic target, 5) as diagnostic markers to specific populations of pain sensing neurons and 6) as targets for structure based ligand identification.

B. Nucleic Acid Molecules

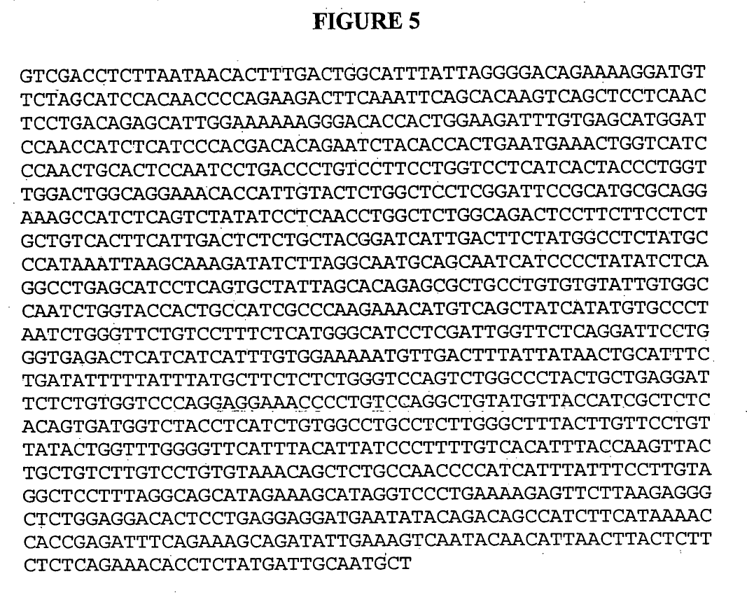

The present invention further provides nucleic acid molecules that encode the MrgC11 proteins having SEQ ID NO: 2 and the related polypeptides herein described, preferably in isolated form. A nucleic acid encoding native murine MrgC11 is provided in FIG. 5 (SEQ ID NO: 1).

Preferred molecules are those that hybridize under the above defined stringent conditions to the complement of SEQ ID NO: 1 and which encode a functional polypeptide. More preferred hybridizing molecules are those that hybridize under the above conditions to the complement strand of the open reading frame or coding sequences of SEQ ID NO: 1 and encode a functional polypeptide.

It is not intended that the methods of the present invention be limited by the source of the polynucleotide. The polynucleotide can be from a human or non-human mammal, derived from any recombinant source, synthesized in vitro or by chemical synthesis. The nucleotide may be DNA or RNA and may exist in a double-stranded, single-stranded or partially double-stranded form.

Nucleic acids useful in the present invention include, by way of example and not limitation, oligonucleotides such as antisense DNAs and/or RNAs; ribozymes; DNA for gene therapy; DNA and/or RNA chimeras; various structural forms of DNA including single-stranded DNA, double-stranded DNA, supercoiled DNA and/or triple-helix DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach, IRL Press, Oxford, England).

Any mRNA transcript encoded by MrgC11 nucleic acid sequences may be used in the methods of the present invention, including in particular, mRNA transcripts resulting from alternative splicing or processing of mRNA precursors.

Nucleic acids having modified nucleoside linkages may also be used in the methods of the present invention. Modified nucleic acids may, for example, have greater resistance to degradation. Such nucleic acids may be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂—CH₂), 2′-O-alkyl, and 2′-deoxy-2′-fluoro phosphorothioate internucleoside linkages are well known in the art.

In some embodiments of the present invention, the nucleotide used is an α-anomeric nucleotide. An α-anomeric nucleotide forms specific double-stranded hybrids with complementary RNA in which, contrary to the usual β-units, the strands run parallel to each other (Gautier et al., 1987, Nucl. Acids Res. 15:6625-6641). The nucleotide may be a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Means for purifying the nucleic acids of the present invention are well known in the art and the skilled artisan will be able to choose the most appropriate method of purification for the particular circumstances. Such a choice may be made, in part, based on the size of the DNA, the amount to be purified and the desired purity. For example, the nucleic acids can be purified by reverse phase or ion exchange HPLC, size exclusion chromatography or gel electrophoresis.

Isolated or purified polynucleotides having at least 10 nucleotides (i.e., a hybridizable portion) of an MrgC11 coding sequence or its complement may also be used in the methods of the present invention. In other embodiments, the polynucleotides contain at least 25 (continuous) nucleotides, 50 nucleotides, 100 nucleotides, 150 nucleotides, or 200 nucleotides of an MrgC11 coding sequence, or a full-length MrgC11 coding sequence. Nucleic acids can be single or double stranded. Additionally, the invention relates to polynucleotides that selectively hybridize to a complement of the foregoing coding sequences. In preferred embodiments, the polynucleotides contain at least 10, 25, 50, 100, 150 or 200 nucleotides or the entire length of an MrgC11 coding sequence.

Nucleotide sequences that encode a mutant of an MrgC11 protein, peptide fragments of MrgC11, truncated forms of MrgC11, and MrgC11 fusion proteins may also be useful in the methods of the present invention. Nucleotides encoding fusion proteins may include, but are not limited to, full length MrgC11 sequences, truncated forms of MrgC11, or nucleotides encoding peptide fragments of MrgC11 fused to an unrelated protein or peptide, such as for example, a domain fused to an Ig Fc domain or fused to an enzyme such as a fluorescent protein or a luminescent protein which can be used as a marker.

Furthermore, polynucleotide variants that have been generated, at least in part, by some form of directed evolution, such as gene shuffling or recursive sequence recombination may be used in the methods of the present invention. For example, using such techniques novel sequences can be generated encoding proteins similar to MrgC11 but having altered functional or structural characteristics.

Highly related gene homologs of the MrgC11 encoding polynucleotide sequences described above may also be useful in the present invention. Highly related homologs can encode proteins sharing functional activities with MrgC11 proteins.

The present invention further provides fragments of the encoding nucleic acid molecule. Fragments of the encoding nucleic acid molecules of the present invention (i.e., synthetic oligonucleotides) that are used as probes or specific primers for the polymerase chain reaction (PCR), or to synthesize gene sequences encoding proteins of the invention, can easily be synthesized by chemical techniques, for example, the phosphotriester method of Matteucci, et al., (J. Am. Chem. Soc. 103:3185-3191, 1981) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention.

Any nucleotide sequence which encodes the amino acid sequence of a protein of the invention can be used to generate recombinant molecules which direct the expression of the protein, as described in more detail below. In addition, the methods of the present invention may also utilize a fusion polynucleotide comprising an MrgC11 coding sequence and a second coding sequence for a heterologous protein.

C. Isolation of Other Related Nucleic Acid Molecules

As described above, the identification and characterization of a nucleic acid molecule encoding MrgC11 allows a skilled artisan to isolate nucleic acid molecules that encode other members of the same protein family, particularly other expressed members of the MrgC family.

A skilled artisan can readily use the amino acid sequence of SEQ ID NO: 2 to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as a lambda gtll library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the protein.

Alternatively, a portion of the coding sequence herein described can be synthesized and used as a probe to retrieve DNA encoding a member of the MrgC protein family from cells derived from any mammalian organism, particularly cells believed to express MrgC proteins, such as DRG cells. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives. Oligonucleotides corresponding to either the 5′ or 3′ terminus of the coding sequence may be used to obtain longer nucleotide sequences.

It may be necessary to screen multiple cDNA libraries to obtain a full-length cDNA. In addition, it may be necessary to use a technique such as the RACE (Rapid Amplification of cDNA Ends) technique to obtain the complete 5′ terminal coding region. RACE is a PCR-based strategy for amplifying the 5′ end of incomplete cDNAs. To obtain the 5′ end of the cDNA, PCR is carried out on 5′-RACE-Ready cDNA using an anchor primer and a 3′ primer. A second PCR is then carried out using the anchored primer and a nested 3′ primer. Once a full length cDNA sequence is obtained, it may be translated into amino acid sequence and examined for identifiable regions such as a continuous open reading frame flanked by translation initiation and termination sites, a potential signal sequence and finally overall structural similarity to the protein sequences disclosed herein.

Related nucleic acid molecules may also be retrieved by using pairs of oligonucleotide primers in a polymerase chain reaction (PCR) to selectively clone an encoding nucleic acid molecule. The oligonucleotide primers may be degenerate oligonucleotide primer pools designed on the basis of the protein coding sequences disclosed herein. The template for the reaction may be cDNA obtained by reverse transcription (RT) of mRNA prepared from, for example, human or non-human cell lines or tissues known or suspected to express an MrgC gene allele, such as DRG tissue. A PCR denature/anneal/extend cycle for using such PCR primers is well known in the art and can readily be adapted for use in isolating other encoding nucleic acid molecules.

The PCR product may be subcloned and sequenced to ensure that the amplified sequences represent the sequences of an MrgC coding sequence. The PCR fragment may then be used to isolate a full-length cDNA clone by a variety of methods. For example, the amplified fragment may be labeled and used to screen a cDNA library. Alternatively, the labeled fragment may be used to isolate genomic clones via the screening of a genomic library.

PCR technology may also be utilized to isolate full-length cDNA sequences. RNA may be isolated, from an appropriate cellular or tissue source, such as dorsal root ganglion (DRG) and an RT reaction may be carried out using an oligonucleotide primer specific for the most 5′ end of the amplified fragment to prime first strand synthesis. The resulting RNA/DNA hybrid may then be “tailed” with guanines in a terminal transferase reaction, the hybrid may be digested with RNAase H, and second strand synthesis may then be primed with a poly-C primer. This allows isolation of cDNA sequences upstream of the amplified fragment.

Nucleic acid molecules encoding other members of the MrgC family may also be identified in existing genomic or other sequence information using any available computational method, including but not limited to: PSI-BLAST (Altschul, et al. (1997) Nucleic Acids Res. 25:3389-3402); PHI-BLAST (Zhang, et al. (1998), Nucleic Acids Res. 26:3986-3990), 3D-PSSM (Kelly et al. J. Mol. Biol. 299(2): 499-520 (2000)); and other computational analysis methods (Shi et al. Biochem. Biophys. Res. Commun. 262(1):132-8 (1999) and Matsunami et. al. Nature 404(6778):601-4 (2000).

A cDNA clone of a mutant or allelic variant of MrgC11 may also be isolated. A possible source of a mutant or variant protein is tissue known to express MrgC11, such as DRG tissue, obtained from an individual putatively carrying a mutant or variant form of MrgC1. Such an individual may be identified, for example, by a demonstration of increased or decreased responsiveness to painful stimuli. In one embodiment, a mutant or variant MrgC11 gene may be identified by PCR. The first cDNA strand may be synthesized by hybridizing an oligo-dT oligonucleotide to mRNA isolated from the tissue putatively carrying a variant and extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant MrgC11 allele to that of the normal MrgC11 allele, the mutation(s) responsible for any loss or alteration of function of the mutant MrgC11 gene product can be ascertained.

Alternatively, a genomic library can be constructed using DNA obtained from an individual suspected of or known to carry a mutant MrgC11 allele, or a cDNA library can be constructed using RNA from a tissue known, or suspected, to express a mutant MrgC11 allele. An unimpaired MrgC11 gene or any suitable fragment thereof may then be labeled and used as a probe to identify the corresponding mutant MrgC11 allele in such libraries. Clones containing the mutant MrgC11 gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant MrgC11 allele in an individual suspected of carrying such a mutant allele. In this manner, gene products made by the putatively mutant tissue may be expressed and screened using standard antibody screening techniques in conjunction with antibodies raised against the normal MrgC11 gene product, as described, below.

D. Recombinant DNA Molecules Containing a Nucleic Acid Molecule

The present invention further provides recombinant DNA molecules (rDNAs) that contain a coding sequence. As used herein, a rDNA molecule is a DNA molecule that has been subjected to molecular manipulation in situ. Methods for generating rDNA molecules are well known in the art, for example, see Sambrook et al., Molecular Cloning: A Laboratory Manual, 2nd edition, 1989; Cold Spring Harbor Laboratory Press, Cold Spring Harbor, N.Y. In the preferred rDNA molecules, a coding DNA sequence is operably linked to expression control sequences and/or vector sequences.

Thus the present invention also contemplates DNA vectors that contain MrgC11 coding sequences and/or their complements, optionally associated with a regulatory element that directs the expression of the coding sequences. The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Both cloning and expression vectors contain a nucleic acid sequence that enables the vector to replicate in one or more selected host cells. In cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

In addition to being capable of replication in at least one class of organism most expression vectors can be transfected into another organism for expression. For example, a vector is replicated in E. coli and then the same vector is transfected into yeast or mammalian cells for expression.

DNA may also be amplified by insertion into the host genome. For example, transfection of Bacillus with a vector comprising a DNA sequence complementary to a Bacillus genomic sequence results in homologous recombination with the genome and insertion of the DNA from the vector. One disadvantage to this type of system is that the recovery of genomic DNA encoding the protein of interest is more complex than that of an exogenously replicated vector because restriction enzyme digestion is required to excise the DNA.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences that are compatible with bacterial hosts are typically provided in plasmid vectors containing convenient restriction sites for insertion of a DNA segment of the present invention. Typical of such vector plasmids are pUC8, pUC9, pBR322 and pBR329 available from BioRad Laboratories, (Richmond, Calif.), pPL and pKK223 available from Pharmacia (Piscataway, N.J.).

Expression vectors compatible with eukaryotic cells, preferably those compatible with vertebrate cells, can also be used to form rDNA molecules that contain a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), eukaryotic viral vectors such as adenoviral or retroviral vectors, and the like eukaryotic expression vectors.

Eukaryotic cell expression vectors used to construct the rDNA molecules of the present invention may further include a selectable marker that is effective in an eukaryotic cell, preferably a drug resistance selection marker. This gene encodes a factor necessary for the survival or growth of transformed host cells grown in a selective culture medium. Host cells not transformed with the vector containing the selection gene will not survive in the culture medium. Typical selection genes encode proteins that confer resistance to antibiotics or other toxins, e.g., ampicillin, neomycin, methotrexate, or tetracycline, complement auxotrophic deficiencies, or supply critical nutrients withheld from the media. A preferred drug resistance marker is the gene whose expression results in neomycin resistance, i.e., the neomycin phosphotransferase (neo) gene. (Southern et al., J. Mol. Anal. Genet. 1:327-341, 1982.) The selectable marker can optionally be present on a separate plasmid and introduced by co-transfection.

In one example of a selection system, mammalian cell transformants are placed under selection pressure such that only the transformants are able to survive by virtue of having taken up the vector(s). Selection pressure is imposed by progressively increasing the concentration of selection agent in the culture medium, thereby stimulating amplification of both the selection gene and the DNA that encodes the desired protein. Amplification is the process by which genes in greater demand for the production of a protein critical for growth are reiterated in tandem within the chromosomes of successive generations of recombinant cells. Increased quantities of the desired protein, such as MrgC11, are synthesized from the amplified DNA. Examples of amplifiable genes include DHFR, thymidine kinase, metallothionein-I and -II, adenosine deaminase, and ornithine decarboxylase.

Thus in one embodiment Chinese hamster ovary (CHO) cells deficient in DHFR activity are prepared and propagated as described by Urlaub et al., Proc. Natl. Acad. Sci. USA, 77:4216 (1980). The CHO cells are then transformed with the DHFR selection gene and transformants are are identified by culturing in a culture medium that contains methotrexate (Mtx), a competitive antagonist of DHFR. The transformed cells are then exposed to increased levels of methotrexate. This leads to the synthesis of multiple copies of the DHFR gene, and, concomitantly, multiple copies of other DNA comprising the expression vectors, such as the DNA encoding the protein of interest, for example DNA encoding MrgC11.

Alternatively, host cells can be transformed or co-transformed with DNA sequences encoding a protein of interest such as MrgC11, wild-type DHFR protein, and another selectable marker such as aminoglycoside 3′-phosphotransferase (APH). The transformants can then be selected by growth in medium containing a selection agent for the selectable marker such as an aminoglycosidic antibiotic, e.g., kanamycin, neomycin, or G418.

As mentioned above, expression and cloning vectors usually contain a promoter that is recognized by the host organism and is operably linked to the nucleic acid encoding the protein of interest. Promoters are untranslated sequences located upstream (5′) to the start codon of a structural gene (generally within about 100 to 1000 bp) and control the transcription and translation of the particular nucleic acid sequence, such as an MrgC11 nucleic acid sequence, to which they are operably linked. Promoters may be inducible or constitutive. Inducible promoters initiate increased levels of transcription from DNA under their control in response to some change in culture conditions, such as a change in temperature. Many different promoters are well known in the art, as are methods for operably linking the promoter to the DNA encoding the protein of interest. Both the native MrgC11 promoter sequence and many heterologous promoters may be used to direct amplification and/or expression of the MrgC11 DNA. However, heterologous promoters are preferred, as they generally permit greater transcription and higher yields of the desired protein as compared to the native promoter.

Promoters suitable for use with prokaryotic hosts include, for example, the β-lactamase and lactose promoter systems (Chang et al., Nature, 275:615 (1978); Goeddel et al., Nature, 281:544 (1979)). However, other bacterial promoters are well known in the art and are suitable. Promoters for use in bacterial systems also will contain a Shine-Delgarno (S.D.) sequence operably linked to the DNA encoding the protein of interest.

Promoter sequences that can be used in eukaryotic cells are also well known. Virtually all eukaryotic genes have an AT-rich region located approximately 25 to 30 bases upstream from the transcription initiation site. Another sequence found 70 to 80 bases upstream from the start of transcription of many genes is a CXCAAT region where X may be any nucleotide. At the 3′ end of most eukaryotic genes is an AATAAA sequence that may be the signal for addition of the poly-A tail to the 3′ end of the coding sequence. All of these sequences may be inserted into eukaryotic expression vectors.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)).

Inducible promoters for use with yeast are also well known and include the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Transcription of MrgC11 from vectors in mammalian host cells may also be controlled by promoters obtained from the genomes of viruses such as polyoma virus, fowlpox virus, adenovirus, bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, and from the promoter normally associated with the native sequence, provided such promoters are compatible with the host cell systems.

Transcription may be increased by inserting an enhancer sequence into the vector. Enhancers are cis-acting elements of DNA, usually about 10 to 300 bp in length, that act on a promoter to increase its transcription. Many enhancer sequences are now known from mammalian genes (globin, elastase, albumin, α-fetoprotein, and insulin). Preferably an enhancer from a eukaryotic cell virus will be used. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the protein-encoding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. These sequences are often found in the 5′ and, occasionally 3′, untranslated regions of eukaryotic or viral DNAs or cDNAs and are well known in the art.

Plasmid vectors containing one or more of the components described above are readily constructed using standard techniques well known in the art.

For analysis to confirm correct sequences in plasmids constructed, the plasmid may be replicated in E. coli, purified, and analyzed by restriction endonuclease digestion, and/or sequenced by conventional methods.

Particularly useful in the preparation of proteins of the present invention are expression vectors that provide for transient expression in mammalian cells of DNA encoding MrgC11. Transient expression involves the use of an expression vector that is able to replicate efficiently in a host cell, such that the host cell accumulates many copies of the expression vector and, in turn, synthesizes high levels of a the polypeptide encoded by the expression vector. Sambrook et al., supra, pp. 16.17-16.22. Transient expression systems allow for the convenient positive identification of polypeptides encoded by cloned DNAs, as well as for the screening of such polypeptides for desired biological or physiological properties. Thus, transient expression systems are particularly useful in the invention for purposes of identifying biologically active analogs and variants of the polypeptides of the invention and for identifying agonists and antagonists thereof.

Other methods, vectors, and host cells suitable for adaptation to the synthesis of MrgC11 in recombinant vertebrate cell culture are well known in the art and are readily adapted to the specific circumstances.

E. Host Cells Containing an Exogenously Supplied Coding Nucleic Acid Molecule

The present invention further provides host cells transformed with a nucleic acid molecule that encodes an MrgC11 protein of the present invention. The host cell can be either prokaryotic or eukaryotic but is preferably eukaryotic.

Eukaryotic cells useful for expression of a protein of the invention are not limited, so long as the cell line is compatible with cell culture methods and compatible with the propagation of the expression vector and expression of the gene product. Such host cells are capable of complex processing and glycosylation activities. In principle, any higher eukaryotic cell culture is workable, whether from vertebrate or invertebrate culture. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH/3T3) available from the ATCC as CRL 1658, baby hamster kidney cells (BHK), HEK293 cells and the like eukaryotic tissue culture cell lines.

Propagation of vertebrate cells in culture is a routine procedure. See, e.g., Tissue Culture, Academic Press, Kruse and Patterson, editors (1973). Additional examples of useful mammalian host cell lines that can be readily cultured are monkey kidney CV1 line transformed by SV40 (COS-7, ATCC CRL 1651); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); and mouse mammary tumor (MMT 060562, ATCC CCL51).

Xenopus oocytes may also be directly injected with RNA capable of expressing MrgC11 by standard procedures (see Tominaga et al. Jpn J. Pharmacol. 83(1):20-4 (2000); Tominaga et al. Neuron 21(3):531-43 (1998) and Bisogno et al. Biochem, Biophys. Res. Commun. 262(1):275-84 (1999)).

Examples of invertebrate cells that can be used as hosts include plant and insect cells. Numerous baculoviral strains and variants and corresponding permissive insect host cells are known in the art and may be utilized in the methods of the present invention. In addition, plant cell cultures are known and may be transfected, for example, by incubation with Agrobacterium tumefaciens, which has been manipulated to contain MrgC11 encoding DNA.

Any prokaryotic host can be used to express a rDNA molecule encoding a protein or a protein fragment of the invention. Suitable prokaryotes include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. The preferred prokaryotic host is E. coli. In addition, it is preferably that the host cell secrete minimal amounts of proteolytic enzymes.

In addition to prokaryotes, eukaryotic microbes such as filamentous fungi or yeast are suitable cloning or expression hosts for MrgC11-encoding vectors. For example, Saccharomyces cerevisiae may be used. In addition a number of other genera, species, and strains are commonly available and useful herein, such as Schizosaccharomyces pombe (Beach et al. Nature, 290:140 (1981); EP 139,383); Kluyveromyces hosts (U.S. Pat. No. 4,943,529; Fleer et al., supra) such as, e.g., K. lactis (MW98-8C, CBS683, CBS4574; Louvencourt et al., J. Bacteriol., 737 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., supra), K. thermotolerans, and K. marxianus; yarrowia (EP 402,226); Pichia pastoris (EP 183,070; Sreekrishna et al. J. Basic Microbiol., 28:265-278 (1988)); Candida; Trichoderma reesia (EP 244,234); Neurospora crassa (Case et al. Proc. Natl. Acad. Sci. USA, 76:5259-5263 (1979)); Schwanniomyces such as Schwanniomyces occidentalis (EP 394,538); and filamentous fungi such as, e.g., Neurospora, Penicillium, Tolypocladium (WO 91/00357), and Aspergillus hosts such as A. nidulans (Ballance et al. Biochem. Biophys. Res. Commun., 112:284-289 (1983); Tilburn et al., Gene, 26:205-221 (1983); Yelton et al. Proc. Natl. Acad. Sci. USA, 81:1470-1474 (1984)) and A. niger (Kelly et al. EMBO J., 4:475-479 (1985)).

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al. Proc. Natl. Acad. Sci. USA 69:2110, (1972); and Maniatis et al., Molecular Cloning, A Laboratory Manual, Cold Spring Harbor Laboratory, Cold Spring Harbor, N.Y. (1982). With regard to transformation of vertebrate cells with vectors containing rDNAs, electroporation, cationic lipid or salt treatment methods are typically employed, see, for example, Graham et al. Virol. 52:456, (1973); Wigler et al. Proc. Natl. Acad. Sci. USA 76:1373-76, (1979). The calcium phosphate precipitation method is preferred. However, other methods of for introducing DNA into cells may also be used, including nuclear microinjection and bacterial protoplast fusion.

For transient expression of recombinant channels, transformed host cells for the measurement of Na⁺ current or intracellular Na⁺ levels are typically prepared by co-transfecting constructs into cells such as HEK293 cells with a fluorescent reporter plasmid (such as pGreen Lantern-1, Life Technologies) using the calcium-phosphate precipitation technique (Ukomadu et al. Neuron 8, 663-676 (1992)). After forty-eight hours, cells with green fluorescence are selected for recording (Dib-Hajj et al. FEBS Lett. 416, 11-14 (1997)). Similarly, for transient expression of MrgC11 receptors and measurement of intracellular Ca²⁺ changes in response to receptor activation, HEK cells can be co-transfected with MrgC11 expression constructs and a fluorescent reporter plasmid. HEK293 cells are typically grown in high glucose DMEM (Life Technologies) supplemented with 10% fetal calf serum (Life Technologies).

Prokaryotic cells used to produce polypeptides of this invention are cultured in suitable media as described generally in Sambrook et al., supra.

The mammalian host cells used to produce the polypeptides of this invention may be cultured in a variety of media, including but not limited to commercially available media such as Ham's F10 (Sigma), Minimal Essential Medium ((MEM), Sigma), RPMI-1640 (Sigma), and Dulbecco's Modified Eagle's Medium ((DMEM), Sigma). In addition, any of the media described in Ham et al. Meth. Enz., 58:44 (1979), Barnes et al. Anal. Biochem. 102:255 (1980), U.S. Pat. No. 4,767,704, 4,657,866, 4,927,762, 4,560,655, or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations as determined by the skilled practitioner. The culture conditions are those previously used with the host cell selected for expression, and will be apparent to the skilled artisan.

The host cells referred to in this disclosure encompass cells in culture as well as cells that are within a host animal.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, J. Mol. Biol. 98:503, (1975), or Berent et al., Biotech. 3:208, (1985) or the proteins produced from the cell assayed via an immunological method as described below.

Gene amplification and/or expression may be measured by any technique known in the art, including Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Various labels may be employed, most commonly radioisotopes, particularly ³²P. Immunological methods for measuring gene expression include immunohistochemical staining of tissue sections or cells in culture, as well as assaying protein levels in culture medium or body fluids. With immunohistochemical staining techniques, a cell sample is prepared by dehydration and fixation, followed by reaction with labeled antibodies specific for the gene product, where the labels are usually visually detectable, such as enzymatic labels, fluorescent labels, luminescent labels, and the like.

Antibodies useful for immunohistochemical staining and/or assay of sample fluids may be either monoclonal or polyclonal, and may be prepared as described herein.

F. Production of Recombinant Proteins using an rDNA Molecule

The present invention further provides methods for producing a protein of the invention using nucleic acid molecules herein described. In general terms, the production of a recombinant form of a protein typically involves the following steps:

A nucleic acid molecule is first obtained that encodes an MrgC11 protein of the invention, for example, nucleotides 160-1128 of SEQ ID NO: 1. If the encoding sequence is uninterrupted by introns, as are these sequences, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated or when the recombinant cells are used, for instance, in high throughput assays.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

In one embodiment, MrgC11 may be produced by homologous recombination. Briefly, primary human cells containing an MrgC11-encoding gene are transformed with a vector comprising an amplifiable gene (such as dihydrofolate reductase (DHFR)) and at least one flanking region of a length of at least about 150 bp that is homologous with a DNA sequence at the locus of the coding region of the MrgC11 gene. The amplifiable gene must be located such that it does not interfere with expression of the MrgC11 gene. Upon transformation the construct becomes homologously integrated into the genome of the primary cells to define an amplifiable region.

Transformed cells are then selected for by means of the amplifiable gene or another marker present in the construct. The presence of the marker gene establishes the presence and integration of the construct into the host genome. PCR, followed by sequencing or restriction fragment analysis may be used to confirm that homologous recombination occurred.

The entire amplifiable region is then isolated from the identified primary cells and transformed into host cells. Clones are then selected that contain the amplifiable region, which is then amplified by treatment with an amplifying agent. Finally, the host cells are grown so as to express the gene and produce the desired protein.

The proteins of this invention may be produced recombinantly not only directly, but also as a fusion polypeptide with a heterologous polypeptide. In one embodiment the heterologous polypeptide may be a signal sequence. In general, the signal sequence may be a component of the vector, or it may be a part of the MrgC11 DNA that is inserted into the vector. The heterologous signal sequence selected preferably is one that is recognized and processed (i.e., cleaved by a signal peptidase) by the host cell. For expression in prokaryotic host cells the signal sequence may be a prokaryotic signal sequence selected, for example, from the group consisting of the alkaline phosphatase, penicillinase, lpp, and heat-stable enterotoxin II leaders. For yeast secretion the native signal sequence may be substituted by, e.g., the yeast invertase leader, a factor leader (including Saccharomyces and Kluyveromyces α-factor leaders, or acid phosphatase leader and the C. albicans glucoamylase leader). In mammalian cell expression any native signal sequence is satisfactory. Alternatively it may be substituted with a signal sequence from related proteins, as well as viral secretory leaders, for example, the herpes simplex gD signal. The DNA for such precursor regions is ligated in reading frame to DNA encoding the mature protein or a soluble variant thereof.

The heterologous polypeptide may also be a marker polypeptide that can be used, for example, to identify the location of expression of the fusion protein. The marker polypeptide may be any known in the art, such as a fluorescent protein. A preferred marker protein is green fluorescent protein (GFP).

G. Modifications of MrgC11 Polypeptides

Covalent modifications of MrgC11 and its variants are included within the scope of this invention. In one embodiment, specific amino acid residues of a polypeptide of the invention are reacted with an organic derivatizing agent. Derivatization with bifunctional agents is useful, for instance, for crosslinking MrgC11 or MrgC11 fragments or derivatives to a water-insoluble support matrix or surface for use in methods for purifying anti-MrgC11 antibodies and identifying binding partners and ligands. In addition, MrgC11 or MrgC11 fragments may be crosslinked to each other to modulate binding specificity and effector function. Many crosslinking agents are known in the art and include, but are not limited to, 1,1-bis(diazoacetyl)-2-phenylethane, glutaraldehyde, N-hydroxysuccinimide esters, bifunctional maleimides such as bis-N-maleimido-1,8-octane and agents such as methyl-3-[(p-azidophenyl)dithio]propioimidate.

Other contemplated modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Modification of the glycosylation patterns of the polypeptides of the invention are also contemplated. Methods for altering the glycosylation pattern of polypeptides are well known in the art. For example, one or more of the carbohydrate moieties found in native sequence MrgC11 may be removed chemically, enzymatically or by modifying the glycosylation site. Alternatively, additional glycosylation can be added, such as by manipulating the composition of the carbohydrate moities directly or by adding glycosylation sites not present in the native sequence MrgC11 by altering the amino acid sequence.

Another type of covalent modification of the polypeptides of the invention comprises linking the polypeptide or a fragment or derivative thereof to one of a variety of nonproteinaceous polymers, e.g., polyethylene glycol (PEG), polypropylene glycol, or polyoxyalkylenes, in the manner set forth in U.S. Pat. Nos. 4,640,835, 4,496,689, 4,301,144, 4,670,417, 4,791,192 or 4,179,337.

The polypeptides of the present invention may also be modified in a way to form a chimeric molecule comprising MrgC11 fused to another, heterologous polypeptide or amino acid sequence.

In one embodiment, such a chimeric molecule comprises a fusion of MrgC11 with a tag polypeptide that provides an epitope to which an anti-tag antibody can selectively bind. The epitope tag is generally placed at the amino- or carboxyl-terminus of the polypeptide. The epitope tag allows for identification of the chimeric protein as well as purification of the chimeric protein by affinity purification using an anti-tag antibody or another type of affinity matrix that binds to the epitope tag. A number of tag polypeptides and their respective antibodies are well known in the art. Well known tags include poly-histidine (poly-his) or poly-histidine-glycine (poly-his-gly) tags; the flue HA tag polypeptide (Field et al., Mol. Cell. Biol., 8:2159-2165 (1988)); the c-myc tag (Evan et al., Molecular and Cellular Biology, 5:3610-3616 (1985)); the Herpes Simplex virus glycoprotein D (gD) tag (Paborsky et al., Protein Engineering, 3(6):547-553 (1990)) and the Flag-peptide (Hopp et al., BioTechnology, 6:1204-1210 (1988)).

In another embodiment, the chimeric molecule comprises a fusion of MrgC11 with an immunoglobulin or a particular region of an immunoglobulin. To produce an immunoadhesin, the polypeptide of the invention or a fragment or specific domain(s) thereof could be fused to the Fc region of an IgG molecule. Typically the fusion is to an immunoglobulin heavy chain constant region sequence. MrgC11-immunoglobulin chimeras for use in the present invention are normally prepared from nucleic acid encoding one or more extracellular domains, or fragments thereof, of MrgC11 fused C-terminally to nucleic acid encoding the N-terminus of an immunoglobulin constant domain sequence. N-terminal fusions are also possible.

While not required in the immunoadhesins of the present invention, an immunoglobulin light chain might be present either covalently linked to an MrgC11-immunoglobulin heavy chain fusion polypeptide, or directly fused to MrgC11. In order to obtain covalent association, DNA encoding an immunoglobulin light chain may be coexpressed with the DNA encoding the MrgC11-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs.

Bispecific immunoadhesins may also be made. Such immunoadhesins may combine an MrgC11 domain and a domain, such as the extracellular domain, from another receptor. Alternatively, the immunoadhesins herein might comprise portions of MrgC11 and a different Mrg receptor, each fused to an immunoglobulin heavy chain constant domain sequence.

In yet another embodiment, the chimeric molecule of the present invention comprises a fusion of MrgC11 or a fragment or domain(s) thereof, with a heterologous receptor or fragment or domain(s) thereof. The heterologous receptor may be a related Mrg family member, or may be completely unrelated. The heterologous protein fused to the MrgC11 protein may be chosen to obtain a fusion protein with a desired ligand specificity or a desired affinity for a particular ligand or to obtain a fusion protein with a desired effector function.

H. Methods of Using MrgC11 as a Molecular or Diagnostic Probe

The sequences and antibodies, proteins and peptides of the present invention may be used as molecular probes for the detection of cells or tissues related to or involved with sensory perception, especially perception of pain. Although many methods may be used to detect the nucleic acids or proteins of the invention in situ, preferred probes include antisense molecules and anti-MrgC11 antibodies.

Probes for the detection of the nucleic acids or proteins of the invention may find use in the identification of the involvement of MrgC11 in particular disease states, such as glaucoma or chronic pain, or in enhanced or inhibited sensory perception. In particular, probes of the present invention may be useful in determining if MrgC11 expression is increased or decreased in patients demonstrating changes in sensory perception, such as in patients with allodynia, hyperalgesia or chronic pain, or patients with a disease or disorder, such as glaucoma. A determination of decreased expression or overexpression of a polypeptide of the invention may be useful in identifying a therapeutic approach to treating the disorder, such as by administering MrgC11 agonists or antagonists. They may also be used to diagnose disorders, particularly disorders relating to pain perception.

Determination of changes in MrgC11 expression levels in animal models of disease states, particularly pain, may also be useful in identifying the types of disorders that might be effectively treated by compounds that modify expression or activity.

Further, the probes of the invention, including antisense molecules and antibodies, may be used to detect the expression of mutant or variant forms of MrgC11. The ability to detect such variants may be useful in identifying the role that the variants play in particular disease states and in the symptoms experienced by particular patients. Identification of the involvement of a variant of MrgC11 in a disease or disorder may suggest a therapeutic approach for treatment of the disease or disorder, such as gene therapy or the administration of agonists or antagonists known to bind the receptor variant.

In addition, probes of the invention may be used to determine the exact expression pattern of MrgC11. As described in Example 1, in situ hybridization with cRNA riboprobes detected mMrgC11 in newborn (FIG. 1B) and adult (FIG. 1C) DRG neurons.

Expression of MrgC11 in subsets of dorsal root ganglia (DRG) neurons are shown in FIG. 1C. MrgC11 is shown to be expressed by IB4⁺ nociceptive neurons. Double labeling technique was used to co-localize IB4 (green) and MrgC11 (red) in DRG neurons. The same DRG sections were subsequently undergone through FITC-conjugated lectin IB4 binding. There is an extensive overlap between MrgC11 and IB4 staining.

Information about the expression patterns of the receptors of the invention in normal tissue and tissue taken from animal models of disease or patients suffering from a disease or disorder will be useful in further defining the biological function of MrgC11 and in tailoring treatment regimens to the specific receptor or combination of receptors involved in a particular disease or disorder.

I. Methods to Identify Binding Partners

As discussed in more detail below, a number of peptides have been identified as ligands for MrgC11. In particular MrgC11 is activated by all invertebrate and vertebrate neuropeptides terminating with either RF(Y)G or RF(Y)a. In order to identify additional new ligands for MrgC11, compounds that bind to MrgC11 may be first identified. Thus, another embodiment of the present invention provides methods of isolating and identifying binding partners or ligands of proteins of the invention. Macromolecules that interact with MrgC11 are referred to, for purposes of this discussion, as “binding partners.”

Receptor binding can be tested using MrgC11 isolated from its native source or synthesized directly. However, MrgC11 obtained by the recombinant methods described above is preferred.

The compounds which may be screened in accordance with the invention include, but are not limited to polypeptides, peptides, including but not limited to members of random peptide libraries; (see, e.g., Lam, K. S. et al., 1991, Nature 354:82-84; Houghten, R. et al., 1991, Nature 354:84-86) and combinatorial chemistry-derived molecular libraries made of D- and/or L-configuration amino acids, phosphopeptides (including, but not limited to members of random or partially degenerate, directed phosphopeptide libraries; see, e.g., Songyang, Z. et al., 1993, Cell 72:767-778), peptide mimetics, antibodies (including, but not limited to, polyclonal, monoclonal, humanized, anti-idiotypic, chimeric or single chain antibodies, FAb, F(abN)₂ and FAb expression library fragments, and epitope-binding fragments thereof), and small organic and inorganic molecules.

The ability of candidate or test compounds to bind MrgC11 can be measured directly or indirectly, such as in competitive binding assays. In competitive binding experiments, the concentration of the test compound necessary to displace 50% of another compound bound to the receptor (IC₅₀) is used as a measure of binding affinity. In these experiments the other compound is preferably a ligand known to bind to the MrgC11 receptor with high affinity, such as γ2-MSH.

A variety of assay formats may be employed, including biochemical screening assays, immunoassays, cell-based assays and protein-protein binding assays, all of which are well characterized in the art. In one embodiment the assay involves anchoring the test compound onto a solid phase, adding the non-immobilized component comprising the MrgC11 receptor, and detecting MrgC11/test compound complexes anchored on the solid phase at the end of the reaction. In an alternative embodiment, MrgC11 may be anchored onto a solid surface, and adding the test compound, which is not anchored. In both situations either the test compound or the MrgC11 receptor is labeled, either directly or indirectly, to allow for identification of complexes. For example, an MrgC11-Ig immunoadhesin may be anchored to a solid support and contacted with one or more test compounds.

Microtiter plates are preferably utilized as the solid phase and the anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface.

Alternatively, a reaction can be conducted in a liquid phase, the reaction products separated from unreacted components, and complexes detected; e.g., using an immobilized antibody specific for either MrgC11 polypeptide, peptide or fusion protein or the test compound to anchor any complexes formed in solution, and a labeled antibody specific for the other component of the possible complex to detect anchored complexes.

In one embodiment of these methods, a protein of the invention or a fragment of a protein of the invention, for instance, an extracellular domain fragment, is mixed with one or more potential binding partners, or an extract or fraction of a cell, under conditions that allow the association of potential binding partners with the protein of the invention. After mixing, peptides, polypeptides, proteins or other molecules that have become associated with a protein of the invention are separated from the mixture. The binding partner that bound to the protein of the invention can then be removed, identified and further analyzed. To identify and isolate a binding partner, the entire MrgC11 protein, for instance a protein comprising the entire amino acid sequence of SEQ ID NO: 2 can be used. Alternatively, a fragment of the MrgC11 polypeptide can be used.

As used herein, a cellular extract refers to a preparation or fraction which is made from a lysed or disrupted cell. The preferred source of cellular extracts will be cells derived from DRG. Alternatively, cellular extracts may be prepared from cells derived from any tissue, including normal human kidney tissue, or available cell lines, particularly kidney derived cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the protein with the binding partner can occur. Alternatively, one or more known compounds or molecules can be mixed with the protein of the invention. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, and/or unbound compounds or molecules, the binding partner can be dissociated from the complex using conventional methods. For example, dissociation can be accomplished by altering the salt concentration or pH of the mixture.

To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins or any other macromolecule.

Alternatively, binding partners may be identified using a Far-Western assay according to the procedures of Takayama et al. Methods Mol. Biol. 69:171-84 (1997) or Sauder et al. J. Gen. Virol. 77(5): 991-6 or identified through the use of epitope tagged proteins or GST fusion proteins.

Binding partners may also be identified in whole cell binding assays that are well known in the art. In one embodiment, MrgC11 is expressed in cells in which it is not normally expressed, such as COS cells. The cells expressing MrgC1 are then contacted with a potential binding partner that has previously been labeled, preferably with radioactivity or a fluorescent marker. The cells are then washed to remove unbound material and the binding of the potential binding partner to the cells is assessed, for example by collecting the cells on a filter and counting radioactivity. The amount of binding of the potential binding partner to untransfected cells or mock transfected cells is subtracted as background.

This type of assay may be carried out in several alternative ways. For example, in one embodiment it is done using cell membrane fractions from cells transfected with MrgC1 or known to express MrgC11, rather than whole cells. In another embodiment purified MrgC11 is refolded in lipids to produce membranes that are used in the assay.

Alternatively, the nucleic acid molecules of the invention can be used in cell based systems to detect protein-protein interactions (see, e.g., WO99/55356). These systems have been used to identify other protein partner pairs and can readily be adapted to employ the nucleic acid molecules herein described.

Any method suitable for detecting protein-protein interactions may be employed for identifying proteins, including but not limited to soluble, transmembrane or intracellular proteins, that interact with MrgC11. Among the traditional methods which may be employed are co-immunoprecipitation, crosslinking and co-purification through gradients or chromatographic columns to identify proteins that interact with MrgC11. For such assays, the MrgC11 component can be a full-length protein, a soluble derivative thereof, a peptide corresponding to a domain of interest, or a fusion protein containing some region of MrgC11.

Methods may be employed which result in the simultaneous identification of genes that encode proteins capable of interacting with MrgC1. These methods include, for example, probing expression libraries, using labeled MrgC11 or a variant thereof.

One method of detecting protein interactions in vivo that may be used to identify MrgC11 binding partners is the yeast two-hybrid system. This system is well known in the art and is commercially available from Clontech (Palo Alto, Calif.).

Briefly, two hybrid proteins are employed, one comprising the DNA-binding domain of a transcription activator protein fused to MrgC11, or a polypeptide, peptide, or fusion protein therefrom, and the other comprising the transcription activator protein's activation domain fused to an unknown target protein. These proteins are expressed in a strain of the yeast Saccharomyces cerevisiae that contains a reporter gene (e.g., HBS or lacZ) whose regulatory region contains the transcription activator's binding site. While either hybrid protein alone cannot activate transcription of the reporter gene, interaction of the two hybrid proteins reconstitutes the functional activator protein and results in expression of the reporter gene, which is detected by an assay for the reporter gene product.

The target protein is preferably obtained from tissue or cells known to express MrgC11, such as DRG cells. For example, a cDNA library prepared from DRG cells may be used.

Binding partners may also be identified by their ability to interfere with or disrupt the interaction of known ligands. Even if they do not activate MrgC11, binding partners that interfere with interactions with known ligands are useful in regulating or augmenting MrgC11 activity in the body and controlling disorders associated with MrgC11 activity (or a deficiency thereof), such as pain.

Compounds that interfere with the interaction between MrgC11 and a known ligand may be identified by preparing a reaction mixture containing MrgC11 or some variant or fragment thereof, and a known binding partner, such as γ2-MSH or another peptide identified in Table 1 below, under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of MrgC11 and its binding partner. Control reaction mixtures are incubated without the test compound. The formation of any complexes between MrgC11 and the binding partner is then detected. The formation of a complex in the control reaction, but not in the reaction mixture containing the test compound indicates that the compound interferes with the interaction of the MrgC11 and the known binding partner. Additionally, complex formation within reaction mixtures containing the test compound and normal MrgC11 protein may also be compared to complex formation within reaction mixtures containing the test compound and a mutant MrgC11. This comparison may be important in those cases wherein it is desirable to identify compounds that specifically disrupt interactions of mutant, or mutated MrgC11, but not the normal proteins.

The order of addition of reactants can be varied to obtain different information about the compounds being tested. For example, test compounds that interfere with the interaction by competition can be identified by conducting the binding reaction in the presence of the test substance. In this case the test compound is added to the reaction mixture prior to, or simultaneously with, MrgC11 and the known binding partner. Alternatively, test compounds that have the ability to disrupt preformed complexes can be identified by adding the test compound to the reaction mixture after complexes have been formed.

In an alternate embodiment of the invention, a preformed complex of MrgC11 and an interactive binding partner is prepared in which either the MrgC11 or its binding partners is labeled, but the signal generated by the label is quenched due to formation of the complex (see, e.g., U.S. Pat. No. 4,109,496 to Rubenstein which utilizes this approach for immunoassays). The addition of a test compound that competes with and displaces one of the species from the preformed complex results in the generation of a signal above background. In this way, test substances which disrupt the interaction can be identified.

Whole cells expressing MrgC11, membrane fractions prepared from cells expressing MrgC11 or membranes containing refolded MrgC11 may be used in the assays described above. However, these same assays can be employed using peptide fragments that correspond to the binding domains of MrgC11 and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding an MrgC11 protein and screening for disruption of binding of a known ligand.

The compounds identified can be useful, for example, in modulating the activity of wild type and/or mutant MrgC11; can be useful in elaborating the biological function of MrgC11 receptors; can be utilized in screens for identifying compounds that disrupt normal MrgC11 receptor interactions or may themselves disrupt or activate such interactions; and can be useful therapeutically.

J. Methods to Identify Agents that Modulate the Expression of a Nucleic Acid.

Another embodiment of the present invention provides methods for identifying agents that modulate the expression of a nucleic acid encoding MrgC11 or another protein involved in a pathway that utilizes MrgC11. These agents may be, but are not limited to, peptides, peptide mimetics, and small organic molecules that are able to gain entry into an appropriate cell (e.g., in the DRG) and affect the expression of a gene. Agents that modulate the expression of MrgC11 or a protein in an MrgC11 mediated pathway may be useful therapeutically, for example to increase or decrease sensory perception, such as the perception of pain, to treat glaucoma, or to increase or decrease wound healing.

Such assays may utilize any available means of monitoring for changes in the expression level of the nucleic acids of the invention. As used herein, an agent is said to modulate the expression of a nucleic acid of the invention, for instance a nucleic acid encoding the protein having the sequence of SEQ ID NO: 2, if it is capable of up- or down-regulating expression of the gene or mRNA levels in a cell.

In one assay format, cell lines that contain reporter gene fusions between the open reading frames and/or the 5′ or 3′ regulatory sequences of a gene of the invention and any assayable fusion partner may be prepared. Numerous assayable fusion partners are known and readily available including the firefly luciferase gene and the gene encoding chloramphenicol acetyltransferase (Alam et al. Anal. Biochem. 188:245-254 (1990)). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding MrgC11.

Additional assay formats may be used to monitor the ability of the agent to modulate the expression of a nucleic acid encoding MrgC11. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al. (Molecular Cloning: A Laboratory Manual, 2nd Ed. Cold Spring Harbor Laboratory Press, 1989).

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al. (Molecular Cloning: A Laboratory Manual, 2nd Ed. Cold Spring Harbor Laboratory Press, NY, 1989) or Ausubel et al. (Current Protocols in Molecular Biology, Greene Publishing Co., NY, 1995).

Hybridization conditions are modified using known methods, such as those described by Sambrook et al. and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or porous glass wafer. The wafer can then be exposed to total cellular RNA or polyA RNA from a sample under conditions in which the affixed sequences will specifically hybridize. Such wafers and hybridization methods are widely available, for example, those disclosed by Beattie (WO 95/11755). By examining for the ability of a given probe to specifically hybridize to an RNA sample from an untreated cell population and from a cell population exposed to the agent, agents which up or down regulate the expression of a nucleic acid encoding MrgC11 are identified.

Hybridization for qualitative and quantitative analysis of mRNAs may also be carried out by using a RNase Protection Assay (i.e., RPA, see Ma et al. Methods 10: 273-238 (1996)). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes, pH 6.4, 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea/polyacrylamide gels for analysis.

In another assay format, products, cells or cell lines are first identified which express MrgC11 gene products physiologically. Cells and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Such cells or cell lines are then transduced or transfected with an expression vehicle (e.g., a plasmid or viral vector) construct comprising an operable non-translated 5′ or 3′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag. Such a process is well known in the art.

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

The probes described above for identifying differential expression of MrgC11 mRNA in response to applied agents can also be used to identify differential expression of MrgC11 mRNA in populations of mammals, for example populations with differing levels of sensory perception. Methods for identifying differential expression of genes are well known in the art. In one embodiment, mRNA is prepared from tissue or cells taken from patients exhibiting altered sensory perception, such as patients experiencing neuropathic pain, or suffering from a disease or disorder in which the MrgC11 receptor may play a role, such as glaucoma, and MrgC11 expression levels are quantified using the probes described above. The MrgC11 expression levels may then be compared to those in other populations to determine the role that MrgC11 expression is playing in the alteration of sensory perception and to determine whether treatment aimed at increasing or decreasing MrgC11 expression levels would be appropriate.

K. Methods to Identify Agents that Modulate Protein Levels or at Least One Activity of MrgC11.

Another embodiment of the present invention provides methods for identifying agents or conditions that modulate protein levels and/or at least one activity of MrgC11, including agonists and antagonists. Such methods or assays may utilize any means of monitoring or detecting the desired activity.

In one format, the relative amounts of a protein of the invention between a cell population that has been exposed to the agent to be tested compared to an unexposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

In another embodiment, animals known to express MrgC11 are subjected to a particular environmental stimulus and any change produced in MrgC11 expression is measured. Transgenic animals, such as transgenic mice, produced to express MrgC11 in a particular location may be used. The environmental stimulus is not limited and may be, for example, exposure to stressful conditions, or exposure to noxious or painful stimuli. Differences in MrgC11 expression levels in response to environmental stimuli may provide insight into the biological role of MrgC11 and possible treatments for diseases or disorders related to the stimuli used.

Antibody probes are prepared by immunizing suitable mammalian hosts in appropriate immunization protocols using the peptides, polypeptides or proteins of the invention if they are of sufficient length, or, if desired, or if required to enhance immunogenicity, conjugated to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co. (Rockford, Ill.), may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein Nature 256:495-497 (1975)) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonals or the polyclonal antisera which contain the immunologically significant portion can be used as antagonists, as well as the intact antibodies. Use of immunologically reactive fragments, such as the Fab, Fab′, of F(ab′)₂ fragments is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin, such as humanized antibodies as discussed in more detail below.

1. Identification of Agonists and Antagonists

The present invention provides for assays to identify compounds that serve as agonists or antagonists of one or more of the biological properties of MrgC11. MrgC11 agonists and antagonists are useful in the prevention and treatment of problems associated with sensory perception, particularly nociception. MrgC11 agonists and antagonists alter sensory perception, particularly the perception of pain. For example, compounds identified as MrgC11 receptor agonists may be used to stimulate MrgC11 receptor activation. In one embodiment MrgC11 agonists are effective in treating mammals suffering from pain by reducing the perception of pain. Compounds that are identified as MrgC11 receptor antagonists may be used, for example, to decrease the effector functions of MrgC11 receptors. This may be useful in cases where the MrgC11 receptors contain a mutation that produces increased responsiveness, or in cases of MrgC11 receptor overexpression. For instance, in one embodiment MrgC11 receptor antagonists are used to increase the sensitivity of mammals to pain where appropriate, such as in diseases involving decreased sensory responsiveness, like some forms of diabetes.

Assays for identifying agonists or antagonists may be done in vitro or in vivo, by monitoring the response of a cell following binding of the ligand to the receptor, for instance, as described in the Examples below. An agonist will produce a cellular response, while an antagonist will have no effect on cellular response but will be capable of preventing cellular response to a known agonist.

a. Small Molecules

Small molecules may have the ability to act as MrgC11 agonists or antagonists and thus may be screened for an effect on a biological activity of MrgC11. Small molecules preferably have a molecular weight of less than 10 kD, more preferably less than 5 kD and even more preferably less than 2 kD. Such small molecules may include naturally occurring small molecules, synthetic organic or inorganic compounds, peptides and peptide mimetics. However, small molecules in the present invention are not limited to these forms. Extensive libraries of small molecules are commercially available and a wide variety of assays are well known in the art to screen these molecules for the desired activity.

Candidate MrgC11 agonist and antagonist small molecules are preferably first identified in an assay that allows for the rapid identification of potential agonists and antagonists. An example of such an assay is a binding assay wherein the ability of the candidate molecule to bind to the MrgC11 receptor is measured, such as those described above. In another example, the ability of candidate molecules to interfere with the binding of a known ligand, for example γ2-MSH, is measured. Candidate molecules that are identified by their ability to bind to MrgC11 or interfere with the binding of known ligands are then tested for their ability to stimulate or inhibit one or more biological activities.

The activity of the proteins of the invention may be monitored in cells expressing MrgC11 by assaying for physiological changes in the cells upon exposure to the agent or agents to be tested. Such physiological changes include but are not limited to an increase in intracellular free calcium and/or the flow of current across the membrane of the cell.

In one embodiment the protein is expressed in a cell that is capable of producing a second messenger response and that does not normally express MrgC11. The cell is then contacted with the compound of interest and changes in the second messenger response are measured. Methods to monitor or assay these changes are readily available. For instance, MrgC11 may be expressed in cells expressing a G protein α subunit that links receptor activation to increases in intracellular calcium [Ca²⁺]ᵢ, which can be monitored at the single cell level using the FURA-2 calcium indicator dye as disclosed in Chandrashekar et al. Cell 100:703-711, (2000) and in the Examples below.

Similar assays may also be used to identify inhibitors or antagonists of MrgC11 activation. For example, cells expressing MrgC11 and capable of producing a quantifiable response to receptor activation are contacted with a known MrgC11 activator and the compound to be tested. In one embodiment, HEK cells expressing MrgC11 are contacted with γ2-MSH and the compound to be tested. The cellular response is measured, in this case an increase in [Ca²⁺]ᵢ. A decreased response compared to the known activator by itself indicates that the compound acts as an inhibitor of activation.

While such assays may be formatted in any manner, particularly preferred formats are those that allow high throughput screening (HTP). In HTP assays of the invention, it is possible to screen thousands of different modulators or ligands in a single day. For instance, each well of a microtiter plate can be used to run a separate assay, for instance an assay based on the ability of the test compounds to modulate receptor activation derived increases in intracellular calcium as described above.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Sites of interest might be peptides within the membrane spanning regions, cytoplasmic and extracellular peptide loops between these transmembrane regions, or selected sequences within the N-terminal extracellular domain or C-terminal intracellular domain. Agents can be rationally selected or rationally designed by utilizing the peptide sequences that make up these sites.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Grant G A. in: Meyers (ed.) Molecular Biology and Biotechnology (New York, VCH Publishers, 1995), pp. 659-664). A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

b. Antibodies

Another class of agents of the present invention are antibodies immunoreactive with epitopes of MrgC11. These antibodies may be human or non-human, polyclonal or monoclonal and may serve as agonist antibodies or neutralizing antibodies. They include amino acid sequence variants, glycosylation variants and fragments of antibodies. Antibody agents are obtained by immunization of suitable mammalian subjects with peptides, containing as antigenic regions, those portions of the protein intended to be targeted by the antibodies. General techniques for the production of such antibodies and the selection of agonist or neutralizing antibodies are well known in the art.

The antibodies of the present invention can be polyclonal antibodies, monoclonal antibodies, chimeric antibodies, humanized antibodies, human antibodies, heteroconjugate antibodies, or antibody fragments. In addition, the antibodies can be made by any method known in the art, including recombinant methods.

MrgC11 agonist and neutralizing antibodies may be preliminarily identified based on their ability to bind the MrgC11 receptor. For example, Western blot techniques well known in the art may be used to screen a variety of antibodies for their ability to bind MrgC11. MrgC11 agonist and neutralizing antibodies are then identified from the group of candidate antibodies based on their biological activity. In one embodiment, MrgC11 agonist antibodies are identified by their ability to induce activation of a second messenger system in cells expressing the MrgC11 protein and comprising a second messenger system, for example as described above and in the Examples. In one embodiment, HEK cells transfected with MrgC11 are contacted with a potential MrgC11 agonist antibody. An increase in intracellular calcium indicates that the antibody is an agonist antibody.

Identification of a neutralizing antibody involves contacting a cell expressing MrgC11 with a known MrgC11 ligand, such as γ2-MSH, and the candidate antibody and observing the effect of the antibody on MrgC11 activation. In one embodiment, MrgC11 receptors expressed in HEK cells are contacted with an MrgC11 ligand such as γ2-MSH and the candidate neutralizing antibody. A decrease in responsiveness to the ligand indicates that the antibody is a neutralizing antibody.

c. Other Antagonists

MrgC11 antagonists are not limited to MrgC11 binding molecules. Other antagonists include variants of a native MrgC11 receptor that retain the ability to bind an endogenous ligand but is not able to mediate a biological response. Soluble receptors and immunoadhesins that bind MrgC11 ligands may also be antagonists, as may antibodies that specifically bind a ligand near its binding site and prevent its interaction with the native receptor. These antagonists may be identified in the assays described above.

d. Computer Modeling

Computer modeling and searching technologies permit identification of compounds, or the improvement of already identified compounds, that can modulate MrgC11 receptor expression or activity. Once an agonist or antagonist is identified, the active sites or regions, such as ligand binding sites, are determined. The active site can be identified using methods known in the art including, for example, by determining the effect of various amino acid substitutions or deletions on ligand binding or from study of complexes of the relevant compound or composition with its natural ligand, such as with X-ray crystallography.

Next, the three dimensional geometric structure of the active site is determined such as by X-ray crystallography, NMR, chemical crosslinking or other methods known in the art. Computer modeling can be utilized to make predictions about the structure where the experimental results are not clear. Examples of molecular modeling systems are the CHARMm and QUANTA programs (Polygen Corporation, Waltham, Mass.). Once a predicted structure is determined, candidate modulating compounds can be identified by searching databases containing compounds along with information on their molecular structure in an effort to find compuonds that have structures capable of interacting with the active site. The compounds found from this search are potential modulators of the activity of the proteins of the present invention and can be tested in the assays described above.

The agonistic or antagonistic activity of test compounds identified in cell based assays as described above can be further elucidated in assays using animals, for example transgenic animals that overexpress MrgC11 as described in more detail below. In one embodiment, the effect of administration of potential MrgC11 antagonists or agonists on the responsiveness of such transgenic animals to sensory stimuli, such as noxious or painful stimuli, is measured. The therapeutic utility of such compounds may be confirmed by testing in these types of experiments or in animal models of particular disorders, for example animal models of neuropathic pain.

L. Uses for Agents that Modulate at Least One Activity of MrgC11.

As shown in the Examples, MrgC11 is expressed in the primary nociceptive sensory neurons of DRG and is activated by particular neuropeptides.

Agents that modulate, up-or-down-regulate the expression of the protein or agents such as agonists or antagonists of at least one activity of the protein may be used to modulate biological and pathologic processes associated with the protein's function and activity. Several agents that activate MrgC11 are identified in the examples, including γ2-MSH. Thus the present invention provides methods to treat pain, including neuropathic pain, and to restore normal sensitivity following injury.

As described in the Examples, expression of MrgC11 is associated with biological processes of nociception. As used herein, an agent is said to modulate a biological or pathological process when the agent alters the degree, severity or nature of the process. For instance, the neuronal transmission of pain signals may be prevented or modulated by the administration of agents which up-regulate, down-regulate or modulate in some way the expression or at least one activity of MrgC11.

The pain that may be treated by the proteins of the present invention and agonists and antagonists thereof, is not limited in any way and includes pain associated with a disease or disorder, pain associated with tissue damage, pain associated with inflammation, pain associated with noxious stimuli of any kind, and neuropathic pain, including pain associated with peripheral neuropathies, as well as pain without an identifiable source. The pain may be subjective and does not have to be associated with an objectively quantifiable behavior or response.

In addition to treating pain, the compounds and methods of the present invention are useful for increasing or decreasing sensory responsiveness. It may be useful to increase responsiveness to stimuli, including noxious stimuli and painful stimuli, for example in some disease states that are characterized by a decreased responsiveness to stimuli, such as in diabetes.

Certain conditions, such as chronic disease states associated with pain and peripheral neuropathies and particularly conditions resulting from a defective MrgC11 gene, can benefit from an increase in the responsiveness to MrgC11 receptor ligands. Thus, these conditions may be treated by increasing the number of functional MrgC11 receptors in cells of patients suffering from such conditions. This could be achieved by increasing the expression of MrgC11 receptor in cells through gene therapy using MrgC11-encoding nucleic acid. This includes both gene therapy, where a lasting effect is achieved by a single treatment, and gene therapy where the increased expression is transient. Selective expression of MrgC11 in appropriate cells may be achieved by using MrgC11 genes controlled by tissue specific or inducible promoters or by producing localized infection with replication defective viruses carrying a recombinant MrgC11 gene, or by any other method known in the art.

In a further embodiment, patients that suffer from an excess of MrgC11, hypersensitivity to MrgC11 ligands or excessive activation of MrgC11 may be treated by administering an effective amount of anti-sense RNA, anti-sense oligodeoxyribonucleotides, or siRNA corresponding to at least a portion of the MrgC11 gene coding region, thereby decreasing expression of MrgC11. They may also be treated by administering an MrgC11 polypeptide, fragment thereof, such as a fragment comprising one or more extracellular domains, or an immunoadhesin comprising a fragment of MrgC11.

As used herein, a subject to be treated can be any mammal, so long as the mammal is in need of modulation of a pathological or biological process mediated by MrgC11. For example, the subject may be experiencing pain or may be anticipating a painful event, such as surgery. The invention is particularly useful in the treatment of human subjects.

In one embodiment the patient is administered an effective amount of a composition of the present invention, such as an MrgC11 protein, peptide fragment, MrgC11 variant, MrgC11 agonist, MrgC11 antagonist, or anti-MrgC11 antibody.

The agents of the present invention can be provided alone, or in combination with other agents that modulate a particular biological or pathological process. For example, an agent of the present invention can be administered in combination with other known drugs or may be combined with analgesic drugs or non-analgesic drugs used during the treatment of pain that occurs in the presence or absence of one or more other pathological processes. As used herein, two or more agents are said to be administered in combination when the two agents are administered simultaneously or are administered independently in a fashion such that the agents will act at the same time.

The agents of the present invention are administered to a mammal, preferably to a human patient, in accord with known methods. Thus the agents of the present invention can be administered via parenteral, subcutaneous, intravenous, intramuscular, intraperitoneal, intracerebrospinal, intra-articular, intrasynovial, intrathecal, transdermal, topical, inhalation or buccal routes. They may be administered continuously by infusion or by bolus injection. Generally, where the disorder permits the agents should be delivered in a site-specific manner. Alternatively, or concurrently, administration may be by the oral route. The dosage administered will be dependent upon the age, health, and weight of the recipient, kind of concurrent treatment, if any, frequency of treatment, and the nature of the effect desired.

The toxicity and therapeutic efficacy of agents of the present invention can be determined by standard pharmaceutical procedures in cell cultures or experimental animals. While agents that exhibit toxic side effects can be used, care should be taken to design a delivery system that targets such compounds to the desired site of action in order to reduce side effects.

While individual needs vary, determination of optimal ranges of effective amounts of each component is within the skill of the art. For the prevention or treatment of disease, the appropriate dosage of agent will depend on the type of disease to be treated, the severity and course of the disease, whether the agent is administered for preventive or therapeutic purposes, previous therapy, the patient's clinical history and response to the agent, and the discretion of the attending physician. Therapeutic agents are suitably administered to the patient at one time or over a series of treatments. Typical dosages comprise 0.1 to 100 μg/kg body wt. The preferred dosages comprise 0.1 to 10 μg/kg body wt. The most preferred dosages comprise 0.1 to 1 μg/kg body wt. For repeated administrations over several days or longer, depending on the condition, the treatment is sustained until a desired suppression of disease symptoms occurs. The progress of this therapy is easily monitored by conventional techniques and assays.

In addition to the pharmacologically active agent, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell. The agent can also be prepared as a sustained-release formulation, including semipermeable matrices of solid hydrophobic polymers containing the protein. The sustained release preparation may take the form of a gel, film or capsule.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination with other therapeutic or diagnostic agents. In certain preferred embodiments, the compounds of this invention are co-administered along with other compounds typically prescribed for these conditions according to generally accepted medical practice. The compounds of this invention can be utilized in vivo, ordinarily in mammals, such as humans, sheep, horses, cattle, pigs, dogs, cats, rats and mice, or in vitro. When used in vivo, the compounds must be sterile. This is readily accomplished by filtration through sterile filtration membranes.

a. Articles of Manufacture

In another embodiment of the invention, an article of manufacture containing materials useful for the treatment of the disorders described above is provided. The article of manufacture comprises a container and a label or package insert(s) on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). In one embodiment, at least one active agent in the composition is an MrgC11 agonist. In other embodiments at least one active agent in the composition is an MrgC11 antagonist. The label or package insert indicates that the composition is used for treating the condition of choice, such as to treat pain, for example to reduce neuropathic pain.

M. Transgenic Animals

Transgenic animals containing mutant, knock-out or modified genes corresponding to MrgC11 sequences are also included in the invention. Transgenic animals are genetically modified animals into which recombinant, exogenous or cloned genetic material has been experimentally transferred. Such genetic material is often referred to as a “transgene”. The nucleic acid sequence of the transgene, in this case a form of SEQ ID NO: 1, may be integrated either at a locus of a genome where that particular nucleic acid sequence is not otherwise normally found or at the normal locus for the transgene. In addition the transgene may encode a non-functional variant. The transgene may consist of nucleic acid sequences derived from the genome of the same species or of a different species than the species of the target animal.

The term “germ cell line transgenic animal” refers to a transgenic animal in which the genetic alteration or genetic information was introduced into a germ line cell, thereby conferring the ability of the transgenic animal to transfer the genetic information to offspring. If such offspring in fact possess some or all of that alteration or genetic information, then they too are transgenic animals.

The alteration or genetic information may be foreign to the species of animal to which the recipient belongs, foreign only to the particular individual recipient, or may be genetic information already possessed by the recipient. In the last case, the altered or introduced gene may be expressed differently than the native gene.

Transgenic animals can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al. Hypertension 22(4):630-633 (1993); Brenin et al. Surg. Oncol. 6(2)99-110 (1997); Tuan (ed.), Recombinant Gene Expression Protocols, Methods in Molecular Biology No. 62, Humana Press (1997)).

A number of recombinant or transgenic mice have been produced, including those which express an activated oncogene sequence (U.S. Pat. No. 4,736,866); express simian SV40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al. Genetics 143(4):1753-1760 (1996)); or, are capable of generating a fully human antibody response (McCarthy The Lancet 349(9049):405 (1997)).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see, e.g., Kim et al. Mol. Reprod. Dev. 46(4): 515-526 (1997); Houdebine Reprod. Nutr. Dev. 35(6):609-617 (1995); Petters Reprod. Fertil. Dev. 6(5):643-645 (1994); Schnieke et al. Science 278(5346):2130-2133 (1997); and Amoah J. Animal Science 75(2):578-585 (1997)).

The method of introduction of nucleic acid fragments into recombination competent mammalian cells can be by any method that favors co-transformation of multiple nucleic acid molecules. Detailed procedures for producing transgenic animals are readily available to one skilled in the art, including the disclosures in U.S. Pat. No. 5,489,743 and U.S. Pat. No. 5,602,307.

It is contemplated that mice lacking an MrgC11 gene, or in which expression of MrgC11 has been increased or decreased will be used in an assay for determining how MrgC11 influences behavior, including sensory responses, particularly responses to painful stimuli. In particular, transgenic mice will be used to determine if MrgC11 mediates the response to a particular type of noxious stimuli, such as mechanical, thermal or chemical. Thus in one embodiment transgenic mice lacking native MrgC11 receptors, or in which MrgC11 receptor expression levels have been modified, will be tested to determine their sensitivity to pressure, temperature, and other noxious stimuli. Assays for determining sensitivity to stimuli are well known in the art. These include, but are not limited to, assays that measure responsiveness to mechanical pain (von Frey hairs or tail pinch), thermal pain (latency to lick or jump in the hot plate assay), chemical pain (latency to lick when a noxious substance such as capsaicin or formalin is injected in the paw), visceral pain (abdominal stretching in response to intraperitoneal injection of acetic acid) and neuropathic pain. For example, mice in which MrgC11 has been deleted will be tested for their responsiveness to a variety of painful stimuli of varying intensity. By determining the sensory responses that are mediated by MrgC11, therapeutic agents known to stimulate or inhibit MrgC11 can be chosen for the treatment of disease states known to involve these types of responses. In addition, therapeutics specifically aimed at treating disorders involving these responses can be developed by targeting MrgC11.

In one embodiment, transgenic mice expressing MrgC11 are produced. The expression pattern of the MrgC11 protein may then be determined and the effect of the expression of the MrgC11 protein on various sensory modalities may be investigated. Further, the efficacy of potential therapeutic agents may be investigated in these mice.

In addition, the effects of changes in the expression levels of MrgC11 can be investigated in animal models of disease states. By identifying the effect of increasing or decreasing MrgC11 receptor levels and activation, therapeutic regimens useful in treating the diseases can be developed. In one embodiment, mice in which MrgC11 receptor expression levels have been increased or decreased are tested in models of neuropathic pain.

Further, mice in which MrgC11 expression levels have been manipulated may be tested for their ability to respond to compounds known to modulate responsiveness to pain, such as analgesics. In this way the role of MrgC11 in the sensation of pain may be further elucidated. For example, a lack of response to a known analgesic in the transgenic mice lacking MrgC11 would indicate that the MrgC11 receptors play a role in mediating the action of the analgesic.

Another preferred transgenic mouse is one in which the MrgC11 gene is coexpressed with a marker or tracer such as green fluorescent protein (GFP). By examining the expression pattern of the marker or tracer, the exact location and projection of MrgC11 containing neurons and other cells can be mapped. This information will be compared to the location and projection of neurons and other cells whose involvement in specific disease states has previously been identified. In this way additional therapeutic uses for the compounds of the present invention may be realized.

N. Diagnostic Methods

MrgC11 genes and proteins may be used to diagnose or monitor the presence or absence of sensory neurons and of biological or pathological activity in sensory neurons. For instance, expression of the genes or proteins of the invention may be used to differentiate between normal and abnormal sensory neuronal activities associated with acute pain, chronic intractable pain, or allodynia. Expression levels can also be used to differentiate between various stages or the severity of neuronal abnormalities. One means of diagnosing pathological states of sensory neurons involved in pain transmission using the nucleic acid molecules or proteins of the invention involves obtaining tissue from living subjects. These subjects may be non-human animal models of pain.

The use of molecular biological tools has become routine in forensic technology. For example, nucleic acid probes may be used to determine the expression of a nucleic acid molecule comprising all or at least part of the sequences of the invention in forensic/pathology specimens. Further, nucleic acid assays may be carried out by any means of conducting a transcriptional profiling analysis. In addition to nucleic acid analysis, forensic methods of the invention may target the proteins of the invention to determine up or down regulation of the genes (Shiverick et al., Biochim Biophys Acta 393(1): 124-33 (1975)).

Methods of the invention may involve treatment of tissues with collagenases or other proteases to make the tissue amenable to cell lysis (Semenov et al., Biull Eksp Biol Med 104(7): 113-6 (1987)).

Assays to detect nucleic acid or protein molecules of the invention may be in any available format. Typical assays for nucleic acid molecules include hybridization or PCR based formats. Typical assays for the detection of proteins, polypeptides or peptides of the invention include the use of antibody probes in any available format such as in situ binding assays, etc. See Harlow et al., Antibodies: A Laboratory Manual, Cold Spring Harbor Laboratory, 1988 and Section G. In preferred embodiments, assays are carried-out with appropriate controls.

The above methods may also be used in other diagnostic protocols, including protocols and methods to detect disease states in other tissues or organs.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1

### Cloning and Expression Analysis of MrgC11

All of the mouse MrgC genes were initially reported to be nonfunctional pseudogenes based on draft mouse genomic sequence data. To determine whether any of the MrgC genes were indeed expressed in DRG neurons, degenerate PCR primers specific for all members of the MrgC subfamily were designed. After PCR amplification from a newborn (P0) DRG cDNA library, sequences corresponding to MrgC11 were identified. No other MrgC gene products were identified, indicating that MrgC11 is the only expressed MrgC gene in the mouse.

A full-length MrgC11 cDNA was cloned from the newborn DRG cDNA library. Contrary to the original prediction that all MrgCs were pseudogenes (Dong et al. Cell 106:619-632 (2001)), this experimentally verified transcript contains an intact ORF that is predicted by hydrophobicity analysis to contain seven transmembrane domains. The MrgC11 protein is 51% and 54% identical to the GPCRs MrgA1 and hMRGX1, respectively (FIG. 1A).

The expression of MrgC11 in newborn and in adult DRG neurons was confirmed by means of in situ hybridization (FIGS. 1B and C). MrgC11 is coexpressed in the small-diameter nociceptive neurons that contain IB-4 binding sites (FIG. 1D).

Nonisotopic in situ hybridization on frozen sections was performed using cRNA probes as previously described (Dong et al., supra). For double labeling with Griffonia simplicifolia IB4 lectin, sections were incubated with 12.5 μg/ml FITC-conjugated IB4 lectin (Sigma) after in situ hybridization. The full-length cDNA-encoding MrgC11 was cloned from a newborn (P0) mouse DRG cDNA library.

### Example 2

### Ligand Identification

To address the questions of the ligand selectivity of the MrgC11 receptor, human embryonic kidney (HEK) 293 cells stably expressing either MrgA1 or MrgC11 were established. Neuropeptides were screened to identify ligands and agonists.

Wild type and Ga knockout (KO) mouse embryonic fibroblasts (MEFs) were prepared and cultured from embryonic day 8.5 to 9.5 embryos as described in Kabarowski et al. (Proc. Natl. Acad. Sci. USA 97:12109-12114 (2000)). HEK293 and Ga KO MEFs were cultured in DMEM/10% FBS. U73122, U73343 and thapsigargin (TG) were purchased from Calbiochem. Fura-2/AM was purchased from Molecular Probes. All other reagents were from Sigma.

HEK293 cells were transfected with cDNA encoding the MrgA1-GFP, mNPFF2-GFP or MrgC11-GFP in pcDNA3.1/Zio(+) plasmid (Invitrogen) using the FuGENE6 transfection reagent (Roche Molecular Biomolecules). The transfected cells were selected with 400 μg/ml zeocin in DMEM supplemented with 10% FBS. Each cloned cell was further selected for membrane localization of receptor-GFP fusion protiens.

The selected cells were maintained in the same medium supplemented with 200 μg/ml zeocin. The stable cell lines were designated HEK-MrgA1, HEK-NPFF2 and HEK-MrgC11. Expression of each receptor was confirmed by Western blotting using an anti-GFP monoclonal antibody (Santa Cruz Biotechnology).

A variety of compounds were tested in a ligand screen to determine whether they act as agonists and elicit receptor specific calcium responses in HEK-MrgA1 and HEKMrgC11 cells.

To identify putative ligands for MrgC11 and MrgA1 receptors, HEK-MrgC11 or HEKMrgA1 stable cell lines were screened in a calcium-mobilization assay using a fluorescence-imaging plate reader (FLEXstation). Briefly, HEK-MrgA1 or HEK-MrgC11 were plated in 96-black-well plates (Corning) and grown to confluence. After incubation with Fura-2/AM for >20 min, cells were washed and equilibrated for 20 min with HBSS (Hanks' balanced salt solution) assay buffer. The fluorescence emission caused by intracellular calcium mobilization elicited by agonists was determined by using a fluorometric imaging plate reader, Flexstation (Molecular Devices). All peptides were from Phoenix Pharmaceuticals (St. Joseph, Mo.), Bachem, American Peptide (Sunnyvale, Calif.), or Sigma.

A panel of known peptides (˜100 peptides) was tested at various concentrations and agonist potencies (EC₅₀) for peptides showing calcium responses were measured (Table 1).

TABLE 1

The EC₅₀ values (in nM) of various peptides for HEK-MrgA1 and
HEK-MrgC11 cells using FLEXstation assay

| | | | |
|---|---|---|---|
| Peptides | Sequences | MrgC11 | MrgA1 |

| | | | | |
|---|---|---|---|---|
| AnthoRF-amide | pEGRFa | (SEQ ID NO:5) | 16 ± 6 | Inactive |
| AF-2 | KHEYLRFa | (SEQ ID NO:6) | 130 ± 24 | Inactive |
| ACEP-1 | SGQSWRPQGRFa | (SEQ ID NO:7) | 46 ± 12 | Inactive |
| FLRF-amide | FLRFa | (SEQ ID NO:8) | 157 ± 12 | 402 ± 21 |
| FMRF-amide | FMRFa | (SEQ ID NO:9) | 114 ± 32 | 420 ± 71 |
| FMRF-OH | FMRF | (SEQ ID NO:10) | 544 ± 117 | 8,204 ± 458 |
| Met-ENK-RFamide | YGGFMRFa | (SEQ ID NO:11) | 133 ± 20 | 5,252 ± 1,280 |
| Met-Enk-RF | YGGFMRF | (SEQ ID NO:12) | 545 ± 19 | Inactive |
| γ1-MSH | YVMGHFRWDRFa | (SEQ ID NO:13) | 17 ± 3 | Inactive |
| γ2-MSH | YVMGHFRWDRFG | (SEQ ID NO: 14) | 11 ± 5 | Inactive |
| BAM3200 YGGFMRRVGRPEWWMDYQKRYGGFL | (SEQ ID NO:15) | 300 ± 124 | >10,000 | |
| BAM-22P | YGGFMRRVGRPEWWMDYQKRYG | (SEQ ID NO:16) | 26 ± 10 | 2,542 ± 654 |
| BAM-15 | VGRPEWWMDYQKRYG | (SEQ ID NO:17) | 53 ± 2 | 23,326 ± 1,866 |
| BAM-15-amide | VGRPEWWMDYQKRYa | (SEQ ID NO:18) | 479 ± 14 | 8,773 ± 493 |
| Dynnorphin-14 | IRPKLKWDNQKRYG | (SEQ ID NO:19) | 22 ± 1 | Inactive |
| PrRP-20 | TPDINPAWYTGRGRIRPVGRFa | (SEQ ID NO:20) | 144 ± 18 | Inactive |
| Kiss(107-121) | KDLPNYNWNSFGLRFa | (SEQ ID NO:21) | 102 ± 24 | Inactive |
| Kiss(112-121) | YNWNSFGLRFa | (SEQ ID NO:22) | 50 ± 4 | Inactive |
| PQRF-amide | PQRFa | (SEQ ID NO:23) | 126 ± 28 | >10,000 |
| NPFF | FLFQPQRFa | (SEQ ID NO:24) | 54 ± 5 | 2,145 ± 245 |
| NPAF | AGEGLNSQFWSLAAPQRFa | (SEQ ID NO:25) | 282 ± 30 | Inactive |
| RFRP-1 | MPHSFANLPLRFa | (SEQ ID NO:26) | 1,245 ± 112 | Inactive |
| RFRP-3 | VPNLPQRFa | (SEQ ID NO:27) | 113 ± 5 | Inactive |
| NPY | YPSKPEDMARYYSALRHYINLITRQRYa | (SEQ ID NO:28) | 237 ± 30 | 3,486 ± 986 |

Data represent means ± SEM from triplicate independent determinations.
Inactive indicates that no activation was detected at concentrations up to 10 mM.

HEK293 parental cells did not respond to peptides shown in Table 1. The neuropeptide γ2-MSH, which is derived from pro-opiomelanocotin (POMC), was the best agonist for MrgC11 (EC₅₀=11±5 nM). However, MrgC11 was not activated by other POMC-derived peptides such as α-MSH, β-MSH, and endorphins (data not shown), which are largely mediated through melanocortin (MC) receptors. On the other hand, FLRFa was found to be the best agonist against MrgA1.

As shown in Table 1, a common feature of all activating peptides for MrgC11 and MrgA1 is the presence of RF(Y)G or RF(Y)a at the C terminus. The invertebrate neuropeptides terminating with −RP or −RN at the C terminus were inactive for both receptors up to 100 μM (data not shown). However, a distinct structure-activity relationship exists between MrgA1 and MrgC11. All peptides comprising an RF(Y)a or RF(Y)G motif at the C terminus were able to activate MrgC11 with different potencies, but only certain peptides among them were able to activate MrgA1 (Table 1). Furthermore, either RFa or RF—OH itself was sufficient to activate MrgC11 with EC₅₀=460±35 nM and 632±124 nM, respectively, whereas RFa or its free acid form was not able to activate MrgA1 (Table 2), suggesting that other as yet unknown structural motifs are required to activate MrgA1 in addition to the RF(Y)a or RF(Y)G motif at the C terminus.

TABLE 2
The EC₅₀ values of FMRFa peptides chirally modified in successive
single residues for HEK-MrgC11 and HEK-MrgA1 cells

| Peptides | MrgC11, nM | MrgA1, nM |
|---|---|---|
| F-M-R-Fa | 114 ± 32 | 420 ± 71 |
| (D)F-M-R-Fa | 108 ± 1 | 882 ± 55 |
| F-(D)M-R-Fa | 11 ± 4 | 1,260 ± 223 |
| F-M-(D)R-Fa | Inactive | Inactive |
| F-M-R-(D)Fa | Inactive | 643 ± 80 |
| R-Fa | 460 ± 35 | Inactive |
| R-F-OH | 632 ± 124 | Inactive |

Data represent mean ± SEM from triplicate independent determinations.

Because the amidation of RFa peptides is known to be critical for agonist activity on RFa receptors, such as GPR54 and NPFF receptors (Bonini et al. J. Biol. Chem. 275:39324-39331 (2000); Muir et al. J. Biol. Chem. 276:28969-28975 (2001); Clements et al. Biochem. Biophys. Res. Commun. 284:1189-1193 (2001)), the effect of amidation and/or deamidation of RFa peptides on the functional affinity for both receptors was measured. The free acid form of FMRFa resulted in about a 20-fold decrease in activity for MrgA1. Also, the deamidated peptide form of YGFMRFa resulted in complete loss of activity for MrgA1, whereas deamidation rendered the peptides about only 4- to 5-fold less active for MrgC11 (Table 1). Inversely, amidation of the BAM-15 peptide caused a modest increase (2.7-fold) in activity for MrgA1, whereas it caused a pronounced decrease (9-fold) for MrgC11. To better define the agonist specificity required for activation of both receptors, the significance of the orientation of the side chains was examined by substituting D-amino acid isomers in each position (Table 2). The change of arginine (Arg) chirality resulted in complete loss of agonist activity for both receptors, suggesting that Arg-3 is a common critical residue (Table 2). Replacement of the Met-2 residue by the D-isomer resulted in a 3-fold decrease in activity for MrgA1, whereas the change resulted in 10-fold increase in activity for MrgC11 (Table 2). This increase might be attributable to an optimization of tertiary structure for better receptor binding. Also, substitution of the Phe-4 with the D-isomer rendered the peptide inactive for MrgC11, whereas it resulted in only slight decrease in activity for MrgA1. These data provide further evidence of structure-activity differences between MrgA1 and MrgC11, though both receptors are activated by RF-amide-related peptides.

### Example 3

### FLRFa and γ2-MSH Elicit Transient Intracellular Calcium Responses in a Receptor-Specific Manner

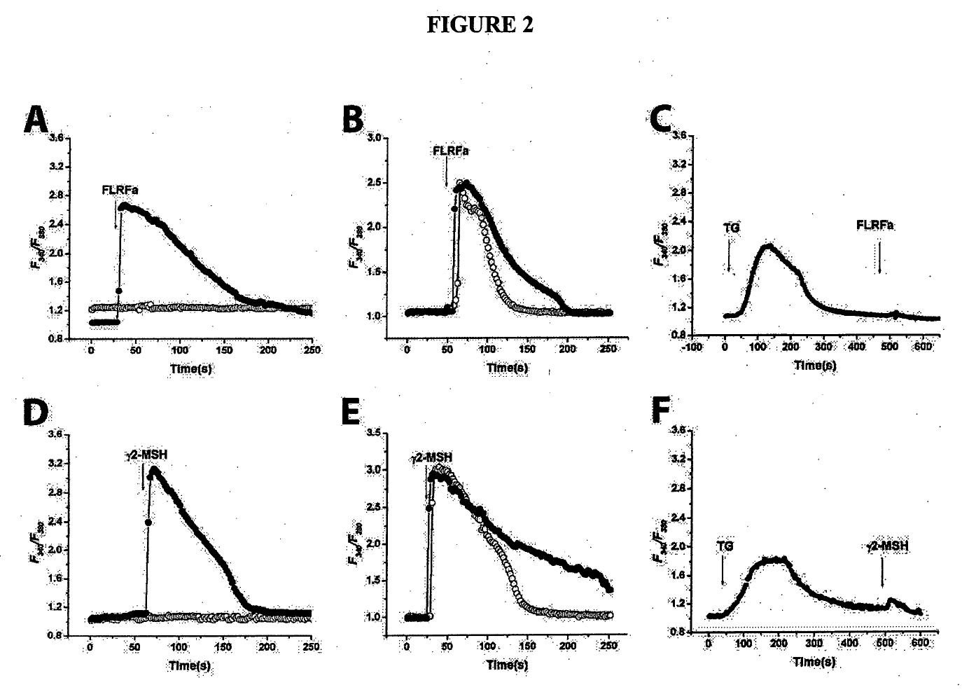

FLRFa or γ2-MSH were used to activate MrgA1 and MrgC11, respectively because these are the most potent agonists amongst the peptides tested for each receptor (see Table 1). Pretreatment of the cells for 10 min with a specific phospholipase C inhibitor, 10 μM U73122 completely inhibited the 3 μM FLRFa or 1 μM γ2-MSH-induced calcium release (FIGS. 2A and D). In contrast, pretreatment of cells with 10 μM U73343 (an inactive analogue of U73122) did not significantly affect [Ca²⁺]ᵢ responses for both receptors (FIGS. 2A and D).

To determine whether Ca²⁺ influx occurs from the extracellular medium, FLRFa- or γ2-MSH-induced [Ca²⁺]ᵢ responses were examined in the presence of 2 mM EGTA (FIGS. 2B and E). In the presence of EGTA, the agonist-induced calcium responses were similar in amplitude to the responses obtained in medium containing the normal level of calcium (FIGS. 2B and E). However, the response rapidly returned to basal levels, suggesting that in the absence of EGTA, Ca²⁺ influx occurred (FIGS. 2B and E).

The calcium source responsible for the initial peak in [Ca²⁺]ᵢ was determined by depleting internal calcium stores with the application of 1 μM TG (FIGS. 2C and F). When HEK-MrgA1 or HEK-MrgC11 cells were treated with 1 μM TG, the resultant emptying of intracellular calcium stores blocked the response to FLRFa or γ2-MSH (FIGS. 2C and F), indicating that FLRFa or γ2-MSH can trigger the mobilization of calcium from IP₃-dependent internal calcium stores, and that the resultant intracellular calcium can induce the influx of extracellular calcium.

### Example 4

### Internalization of MrgA1 and MrgC11

The ability of agonists to induce the internalization of MrgA1 or MrgC11 was measured, as receptor internalization is a response of GPCRs to ligand stimulation. This process indicates that the agonist interacts directly with its cognate receptor.

Briefly, MrgC11-GFP or MrgA1-GFP stably expressing HEK293 cells were grown in 35 mm glass-bottomed dishes (Mat-Tek, Ashland, Mass.) in DMEM with 10% FBS. After 4-6 hours of serum starvation, cells were treated with agonists at 37° C. for 30 minutes. Cells were washed with PBS and fixed with 3.7% paraformaldehyde in PBS. The subcellular localization of Mrg-GFP was visualized under a Leica confocal fluorescence microscope with a ×20 or ×40 lens.

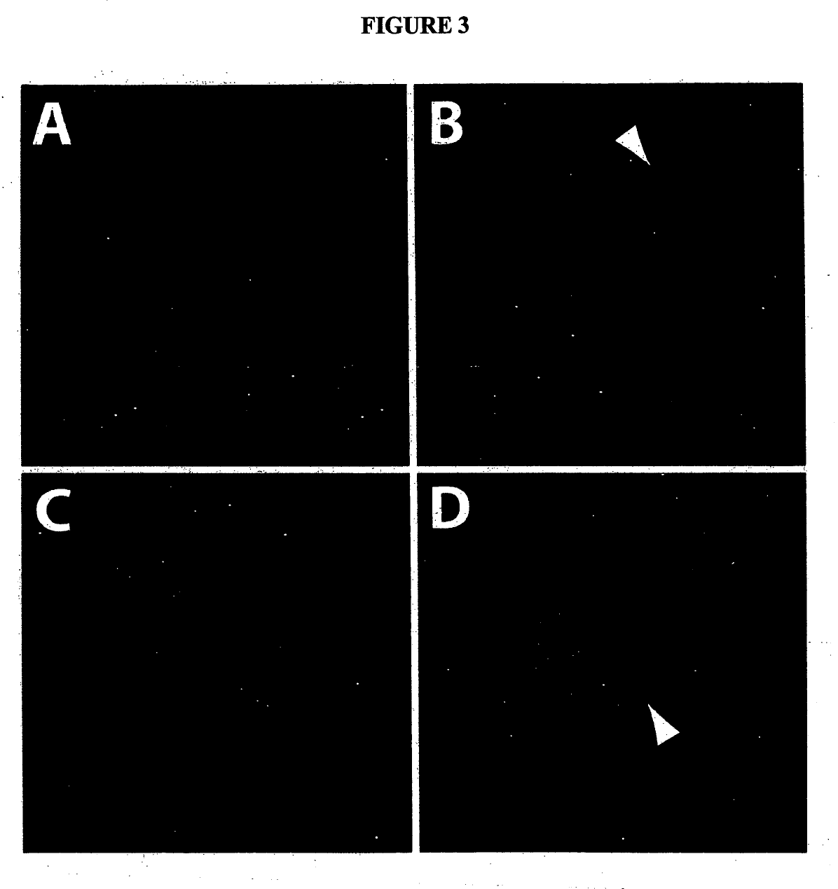

In non-stimulated conditions, MrgA1-GFP or MrgC11-GFP fusion proteins were expressed predominantly at the plasma membrane (FIGS. 3A and C). Stimulation of FLRFa or γ2-MSH induced internalization of MrgA1-GFP (FIG. 3B) or MrgC11-GFP (FIG. 3D) in >90% of cells at 37° C. However, rapid internalization was not observed at room temperature under the same conditions.

### Example 5

### MrgA1 and MrgC11 Coupling to Heterotrimeric G Proteins

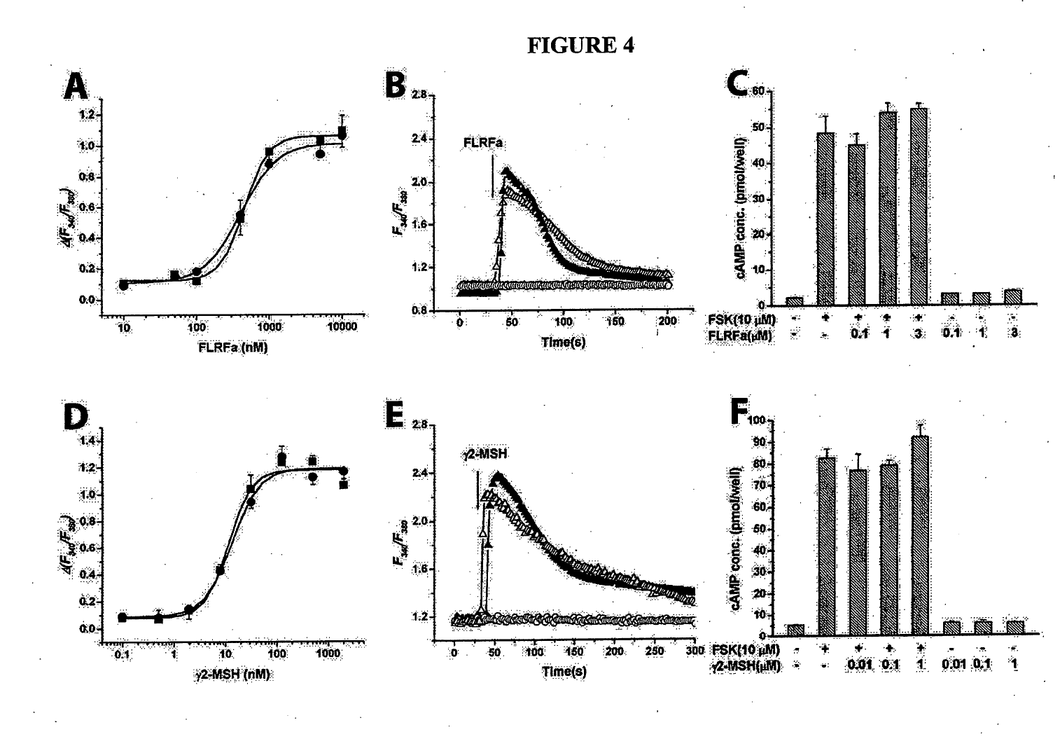

Transiently overexpressed MrgA1 was previously reported to respond to FLRFa with high potency (EC₅₀≈20 nM) in HEK293 cells expressing Gα₁₅. We reexamined the dose dependence in HEK293 cells stably expressing MrgA1 (HEK-MrgA1) but not expressing exogenous Gα₁₅. FLRFa stimulated an increase in [Ca²⁺]ᵢ with an EC₅₀ of 402±21 nM in this cellular system (FIG. 4A). The difference in EC₅₀ value is possibly derived from a variety of sources such as different coupling efficiencies, different expression levels of receptor, and/or different cellular environment. Nonetheless, the relative ligand selectivity (FLRFa vs. NPFF) was conserved in both cellular systems.

Heterotrimeric G proteins of the Gαᵢ and Gα_{q} class are involved in the propagation of signals from GPCRs leading to [Ca²⁺]ᵢ elevation (Guderman et al. Ann. Rev. Pharmacol. Toxicol. 36:429-459 (1996)). To determine whether Gα_{i/o} proteins are involved in the [Ca²⁺]ᵢ response, we pretreated HEK-MrgA1 or HEK-MrgC11 cells with PTX (100 ng/ml) for 16 h. PTX blocks responses mediated by the Gα_{i/o} system of G protein transducers but does not effect signals transmitted through Gas, Gα_{12/13}, or the Gα_{q/11} family. The dose dependency in [Ca²⁺]ᵢ responses for both receptors were not affected by PTX (FIGS. 4A and D). In contrast, PTX completely blocked FLRFa-induced calcium response in HEK-mNPFF2 cells (data not shown).

MEF cell lines derived from Gα_{q/11} or Gα_{12/13} double gene KO mice were used to test whether activation of MrgA1 or MrgC11 receptors can mobilize calcium responses through the direct participation of Gα_{q/11} The Gα_{q/11} KO MEFs or Gα_{12/13} KO MEFs were transfected with cDNAs encoding either MrgA1-GFP or MrgC11-GFP receptor, and the ability of agonists to increase [Ca²⁺]ᵢ was measured in individual cells. The GFP receptor fusion proteins were used to identify positively transfected cells, and single-cell calcium assays were performed as described in Dong et al., supra. Briefly, MrgA1-GFP or MrgC11-GFP-transfected cells were grown in specialized glass-bottom dishes (Bioptechs, Butler, Pa.) and loaded with fura-2/AM in Hepes-buffered saline. By using a dual wavelength spectrofluorometer coupled to an inverted fluorescence microscope, GFP-positive cells were identified by using an excitation wavelength of 488 nm, a dichroic 505 nm long-pass filter, and an emitter filter at a band pass of 535 nm (Chroma Technology, Brattleboro, Vt.). Measurements of [Ca²⁺]ᵢ were performed on individual Mrg-GFP positive cells at excitation wavelength of 340 and 380 nm and an emission wavelength of 510 nm.

FLRFa or γ2-MSH induced robust, transient calcium responses in Gα_{12/13} KO cells expressing MrgA1 or MrgC11, but Gα_{q/11} double KO MEFs failed to respond to FLRFa or γ2-MSH (FIGS. 4B and E). The calcium response in Gα_{q/11} KO cells was rescued when Gα_{q/11} KO cells were cotransfected with plasmids encoding wild-type Gα_{q} and each receptor (FIGS. 4B and E). These observations demonstrated that Gα_{q/11} proteins are coupled to both receptors in the calcium-signaling pathway.

It is also possible that these receptors are coupled to the Gα_{i/o} or to the Gαₛ family of heterotrimeric G proteins. Thus, cAMP production was measured in the presence of various concentrations of agonists and presence or absence of forskolin.

A radioimmuno assay kit (Amersham Pharmacia) was used to measure cAMP. HEK-MrgA1 or HEK-MrgC11 were cultured in 6-well plates coated with matriGel for ˜26 h at 37° C. in growth medium. After 4-6 h serum starvation, cells were stimulated with or without representative agonists in the presence or absence of 10 μM forskolin for 10 minutes. The cells were rapidly washed twice with PBS containing 200 μM Ro20-1724 and cAMP was extracted with 2 ml of cold 60% ethanol. Quantitation of cAMP was then performed by using a [³H] cAMP displacement assay as described in Gilman et al. (Proc. Natl. Acad. Sci. USA 67:305-312 (1970)).

No significant inhibition or activation was observed in the presence of various concentrations of FLRFa or γ2-MSH (FIGS. 4C and F). FLRFa and γ2-MSH were unable to inhibit forskolin-induced cAMP accumulation in these cells (FIGS. 4C and F). Taken together, these results demonstrate that both MrgA1 and MrgC11 are coupled to Gα_{q/11}, but not to Gα_{i/o}, or Gαₛ.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1. An isolated nucleic acid molecule comprising a sequence having at least 90% sequence identity to a nucleic acid molecule that encodes the MrgC11 polypeptide of SEQ ID NO: 2.

2. The isolated nucleic acid molecule of claim 1 operably linked to an expression control element.

3. The isolated nucleic acid molecule of claim 2 operably linked to a promoter element.

4. A vector comprising the isolated nucleic acid molecule of claim 3.

5. A host cell comprising the vector of claim 4.

6. The host cell of claim 5 wherein said host cell is a eukaryotic cell.

7. The host cell of claim 6 wherein said host cell is a hamster embryonic kidney (HEK) cell.

8. A method for producing an MrgC11 polypeptide comprising culturing the host cell of claim 6 under conditions in which the protein encoded by said nucleic acid is expressed.

9. The host cell of claim 5, wherein said host cell is capable of producing a second messenger response.

10. A method for identifying MrgC11 agonists and antagonists comprising the steps of:
a) culturing the host cell of claim 9 under conditions such that the protein encoded by said nucleic acid is expressed;
b) contacting the host cell with one or more test compounds; and
c) measuring the second messenger response in the host cell.

11. The method of claim 10, wherein the test compounds are selected from the group consisting of peptides, peptide mimetics, antibodies, small organic molecules and small inorganic molecules.

12. The method of claim 10, wherein measuring a second messenger response comprises measuring a change in intercellular calcium concentration.

13. The method of claim 12, wherein said change in intercellular calcium concentration is measured with FURA-2 calcium indicator dye.

14. The method of claim 10, additionally comprising identifying compounds that increase the measured second messenger response as agonists.

15. The method of claim 10, additionally comprising contacting the host cell with a peptide ligand after culturing the host cell and prior to contacting the host cell with one or more test compounds.

16. The method of claim 15, additionally comprising identifying compounds that alter the second messenger response to the peptide ligand as antagonists.

17. The method of claim 15, wherein the peptide ligand is selected from the group consisting of γ2-MSH, anthoRF-amide, γ1-MSH, Dynorphin-14 and BAM22P.

18. An isolated nucleic acid molecule comprising a sequence having at least 95% sequence identity to a nucleic acid molecule that encodes the MrgC11 polypeptide of SEQ ID NO: 2.

19. An isolated nucleic acid molecule that encodes a protein having the amino acid sequence of SEQ ID NO: 2.

20. The isolated nucleic acid molecule of claim 18, wherein said nucleic acid molecule comprises the nucleotide sequence of SEQ ID NO: 1.


SEQUENCE LISTING (28 sequences)

SEQ ID NO: 1 - DNA, 1186 bp, Mus musculus
gtcgacctcttaataacactttgactggcatttattaggggacagaaaaggatgttctagcatccacaaccccagaagacttcaaattcagcacaagtcagctcctcaactcctgacagagcattggaaaaaagggacaccactggaagatttgtgagcatggatccaaccatctcatcccacgacacagaatctacaccactgaatgaaactggtcatcccaactgcactccaatcctgaccctgtccttcctggtcctcatcactaccctggttggactggcaggaaacaccattgtactctggctcctcggattccgcatgcgcaggaaagccatctcagtctatatcctcaacctggctctggcagactccttcttcctctgctgtcacttcattgactctctgctacggatcattgacttctatggcctctatgcccataaattaagcaaagatatcttaggcaatgcagcaatcatcccctatatctcaggcctgagcatcctcagtgctattagcacagagcgctgcctgtgtgtattgtggccaatctggtaccactgccatcgcccaagaaacatgtcagctatcatatgtgccctaatctgggttctgtcctttctcatgggcatcctcgattggttctcaggattcctgggtgagactcatcatcatttgtggaaaaatgttgactttattataactgcatttctgatatttttatttatgcttctctctgggtccagtctggccctactgctgaggattctctgtggtcccaggaggaaacccctgtccaggctgtatgttaccatcgctctcacagtgatggtctacctcatctgtggcctgcctcttgggctttacttgttcctgttatactggtttggggttcatttacattatcccttttgtcacatttaccaagttactgctgtcttgtcctgtgtaaacagctctgccaaccccatcatttatttccttgtaggctcctttaggcagcatagaaagcataggtccctgaaaagagttcttaagagggctctggaggacactcctgaggaggatgaatatacagacagccatcttcataaaaccaccgagatttcagaaagcagatattgaaagtcaatacaacattaacttactcttctctcagaaacacctctatgattgcaatgct

SEQ ID NO: 2 - protein, 322 aa, Mus musculus
MDPTISSHDTESTPLNETGHPNCTPILTLSFLVLITTLVGLAGNTIVLWLLGFRMRRKAISVYILNLALADSFFLCCHFIDSLLRIIDFYGLYAHKLSKDILGNAAIIPYISGLSILSAISTERCLCVLWPIWYHCHRPRNMSAIICALIWVLSFLMGILDWFSGFLGETHHHLWKNVDFIITAFLIFLFMLLSGSSLALLLRILCGPRRKPLSRLYVTIALTVMVYLICGLPLGLYLFLLYWFGVHLHYPFCHIYQVTAVLSCVNSSANPIIYFLVGSFRQHRKHRSLKRVLKRALEDTPEEDEYTDSHLHKTTEISESRY

SEQ ID NO: 3 - protein, 304 aa, Mus musculus
MDNTIPGGINITILIPNLMIIIFGLVGLTGNGIVFWLLGFCLHRNAFSVYILNLALADFFFLLGHIIDSILLLLNVFYPITFLLCFYTIMMVLYIAGLSMLSAISTERCLSVLCPIWYHCHRPEHTSTVMCAVIWVLSLLICILNSYFCGFLNTQYKNENGCLALNFFTAAYLMFLFVVLCLSSLALVARLFCGTGQIKLTRLYVTIILSILVFLLCGLPFGIHWFLLFKIKDDFHVFDLGFYLASVVLTAINSCANPIIYFFVGSFRHRLKHQTLKMVLQNALQDTPETAKIMVEMSRSKSEP

SEQ ID NO: 4 - protein, 322 aa, Homo sapiens
MDPTISTLDTELTPINGTEETLCYKQTLSLTVLTCIVSLVGLTGNAVVLWLLGCRMRRNAFSIYILNLAAADFLFLSGRLIYSLLSFISIPHTISKILYPVMMFSYFAGLSFLSAVSTERCLSVLWPIWYRCHRPTHLSAVVCVLLWALSLLRSILEWMLCGFLFSGADSAWCQTSDFITVAWLIFLCVVLCGSSLVLLIRILCGSRKIPLTRLYVTILLTVLVFLLCGLPFGIQFFLFLWIHVDREVLFCHVHLVSIFLSALNSSANPIIYFFVGSFRQRQNRQNLKLVLQRALQDASEVDEGGGQLPEEILELSGSRLEQ

SEQ ID NO: 5 - protein, 4 aa, Artificial Sequence
  AnthoRF-amide peptide.
XGRF

SEQ ID NO: 6 - protein, 7 aa, Artificial Sequence
  AF-2 peptide.
KHEYLRF

SEQ ID NO: 7 - protein, 11 aa, Artificial Sequence
  ACEP-1 peptide.
SGQSWRPQGRF

SEQ ID NO: 8 - protein, 4 aa, Artificial Sequence
  FLRF-amide peptide.
FLRF

SEQ ID NO: 9 - protein, 4 aa, Artificial Sequence
  FMRF-amide peptide.
FMRF

SEQ ID NO: 10 - protein, 4 aa, Artificial Sequence
  FMRF-OH peptide.
FMRF

SEQ ID NO: 11 - protein, 7 aa, Artificial Sequence
  Met-ENK-RFamide peptide.
YGGFMRF

SEQ ID NO: 12 - protein, 7 aa, Artificial Sequence
  Met-ENK-RF peptide.
YGGFMRF

SEQ ID NO: 13 - protein, 11 aa, Artificial Sequence
  gamma1-MSH peptide.
YVMGHFRWDRF

SEQ ID NO: 14 - protein, 12 aa, Artificial Sequence
  gamma2-MSH peptide.
YVMGHFRWDRFG

SEQ ID NO: 15 - protein, 25 aa, Artificial Sequence
  BAM3200 peptide.
YGGFMRRVGRPEWWMDYQKRYGGFL

SEQ ID NO: 16 - protein, 22 aa, Artificial Sequence
  BAM22P peptide.
YGGFMRRVGRPEWWMDYQKRYG

SEQ ID NO: 17 - protein, 15 aa, Artificial Sequence
  BAM-15 peptide.
VGRPEWWMDYQKRYG

SEQ ID NO: 18 - protein, 14 aa, Artificial Sequence
  BAM-15-amide peptide.
VGRPEWWMDYQKRY

SEQ ID NO: 19 - protein, 14 aa, Artificial Sequence
  Dynnorphin-14 peptide.
IRPKLKWDNQKRYG

SEQ ID NO: 20 - protein, 20 aa, Artificial Sequence
  PrRP-20 peptide.
TPDINPAWYTGRGIRPVGRF

SEQ ID NO: 21 - protein, 15 aa, Artificial Sequence
  Kiss(107-121) peptide.
KDLPNYNWNSFGLRF

SEQ ID NO: 22 - protein, 10 aa, Artificial Sequence
  Kiss(112-121) peptide.
YNWNSFGLRF

SEQ ID NO: 23 - protein, 4 aa, Artificial Sequence
  PQRF-amide peptide.
PQRF

SEQ ID NO: 24 - protein, 8 aa, Artificial Sequence
  NPFF peptide.
FLFQPQRF

SEQ ID NO: 25 - protein, 18 aa, Artificial Sequence
  NPAF peptide.
AGEGLNSQFWSLAAPQRF

SEQ ID NO: 26 - protein, 12 aa, Artificial Sequence
  RFRP-1 peptide.
MPHSFANLPLRF

SEQ ID NO: 27 - protein, 8 aa, Artificial Sequence
  RFRP-3 peptide.
VPNLPQRF

SEQ ID NO: 28 - protein, 27 aa, Artificial Sequence
  NPY peptide.
YPSKPEDMARYYSALRHYINLITRQRY